(19) United States
(12) Patent Application Publication
(10) Pub. No.: US 2022/0079212 A1
(43) Pub. Date: Mar. 17, 2022

(54) ALGINATE-BASED SUBSTRATES

(71) Applicant: NICOVENTURES TRADING LIMITED, London (GB)

(72) Inventors: Caroline W.H. Clark, High Point, NC (US); Luis Monsalud, JR., Kernersville, NC (US)

(21) Appl. No.: 17/471,044

(22) Filed: Sep. 9, 2021

Related U.S. Application Data
(60) Provisional application No. 63/077,064, filed on Sep. 11, 2020.

(51) Int. Cl. A24B 15/40; A24D 1/20; A24D 1/22; A24B 13/00; A24B 15/28; A24B 15/167

(52) U.S. Cl. CPC A24B 15/403; A24D 1/20; A24D 1/22; A24B 15/287; A24B 15/284; A24B 15/167; A24B 13/00

(57) ABSTRACT

The present disclosure provides various components entrapped within a cross-linked alginate matrix and products including such component-containing, alginate-based matrices. The disclosure also includes a method for entrapping the components, including mixing the component or components with alginate in water, contacting the mixture with a cation to cross-link the alginate, and removing at least a portion of the water therefrom. The resulting component-containing alginate-based material can then be incorporated within various products, e.g., consumable products such as aerosol-generating devices and components, oral products, and conventional smoking articles.

### CROSS-REFERENCE TO RELATED APPLICATIONS

The present application claims priority to U.S. Provisional Patent Application No. 63/077,064, filed Sep. 11, 2020, which is incorporated by reference herein in its entirety.

### FIELD OF THE DISCLOSURE

The present disclosure relates to aerosol generating components, aerosol delivery devices, and aerosol delivery systems that utilize electrically-generated heat or combustible ignition sources to heat aerosol forming materials, preferably without significant combustion, in order to provide an inhalable substance in the form of an aerosol for human consumption.

### BACKGROUND

Many smoking articles have been proposed through the years as improvements upon, or alternatives to, smoking products based upon combusting tobacco for use. Some example alternatives have included devices wherein a solid or liquid fuel is combusted to transfer heat to tobacco or wherein a chemical reaction is used to provide such heat source. Additional example alternatives use electrical energy to heat tobacco and/or other aerosol generating substrate materials, such as described in U.S. Pat. No. 9,078,473 to Worm et al., which is incorporated herein by reference in its entirety.

The point of the improvements or alternatives to smoking articles typically has been to provide the sensations associated with cigarette, cigar, or pipe smoking, without delivering considerable quantities of incomplete combustion and pyrolysis products. To this end, there have been proposed numerous smoking products, flavor generators, and medicinal inhalers which utilize electrical energy to vaporize or heat a volatile material, or attempt to provide the sensations of cigarette, cigar, or pipe smoking without burning tobacco to a significant degree. See, for example, the various alternative smoking articles, aerosol delivery devices and heat generating sources set forth in the background art described in U.S. Pat. No. 7,726,320 to Robinson et al.; and U.S. Pat. App. Pub. Nos. 2013/0255702 to Griffith, Jr. et al.; and 2014/0096781 to Sears et al., each of which are incorporated herein by reference in their entireties.

Articles that produce the taste and sensation of smoking by electrically heating tobacco, tobacco-derived materials, or other plant derived materials have suffered from inconsistent performance characteristics. For example, some articles have suffered from inconsistent release of flavors or other inhalable materials, inadequate loading of aerosol forming materials on substrates, or the presence of poor sensory characteristics. Accordingly, it can be desirable to provide a smoking article that can provide the sensations of cigarette, cigar, or pipe smoking, that does so without combusting the substrate material and that does so with advantageous performance characteristics.

### BRIEF SUMMARY

The present disclosure relates generally to component-containing, alginate-based substrates comprising an alginate matrix and one or more components entrapped therein (referred to herein as component-containing, alginate-based substrates) and methods of providing and using such component-containing, alginate-based substrates. The components can vary and include, but are not limited to, flavorants and other volatile (and non-volatile) compounds that may advantageously be temporarily entrapped. For example, the component-containing, alginate-based substrates can be employed within consumable products, including products configured for combustible aerosol delivery, products configured for non-combustible aerosol delivery, or products configured for aerosol-free delivery such that the components stay entrapped within the articles/devices during production and storage, and can be released during use.

In one aspect, the disclosure provides a method for providing a composition with a releasable entrapment of one or more components in a component-containing, cross-linked alginate structure, comprising: mixing the one or more components and alginate in water to give a mixture; contacting the mixture with a divalent or trivalent cation to crosslink the alginate, thereby trapping the one or more components within a cross-linked matrix; and removing at least a portion of the water from the cross-linked matrix to give a component-containing alginate structure.

In another aspect is provided a component-containing cross-linked alginate structure, comprising one or more components entrapped within a cross-linked alginate matrix.

In some embodiments, the one or more components are selected from the group consisting of flavorants, sweeteners, aerosol-forming agents, humectants, fillers, preservatives, tobacco materials, and combinations thereof. For example, in certain embodiments, the one or more components comprise a flavorant selected from the group consisting of alcohols, aldehydes, aromatic hydrocarbons, ketones, esters, terpenes, terpenoids, trigeminal sensates, and combinations thereof. In certain embodiments, the flavorant is selected from the group consisting of vanillin, ethyl vanillin, p-anisaldehyde, hexanal, furfural, isovaleraldehyde, cuminaldehyde, benzaldehyde, citronellal, 1-hydroxy-2-propanone, 2-hydroxy-3-methyl-2-cyclopentenone-1-one, allyl hexanoate, ethyl heptanoate, ethyl hexanoate, isoamyl acetate, 3-methylbutyl acetate, sabinene, limonene, gamma-terpinene, beta-farnesene, nerolidol, thujone, myrcene, geraniol, nerol, citronellol, linalool, eucalyptol, and combinations thereof. In certain embodiments, the one or more components comprise a flavorant selected from cream, tea, coffee, fruit, maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, Yerba santa, Bacopa monniera, Gingko biloba, Withania somnifera, cinnamon, sandalwood, jasmine, cascarilla, cocoa, licorice, and combinations thereof.

In some embodiments, the one or more components comprise a plant extract. For example, in certain embodiments, the plant extract is a tobacco extract. In some embodiments, the one or more components comprise an active ingredient, selected from the group consisting of a nicotine component, a botanical/herbal ingredient, a stimulant, an amino acid, a vitamin, an antioxidant, a cannabinoid, a cannabimimetic, a terpene, a pharmaceutical ingredient, and any combination thereof. Examples of active ingredients include, but are not limited to, active ingredients selected from the group consisting of hemp, guarana, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, maca, tisanes, lemon balm, ginseng, star anise, caffeine, theacrine, theobromine, theophylline, GABA, theanine, taurine, Vitamin B6, Vitamin B12, Vitamin E, Vitamin C, cannabidiol (CBD), tetrahydrocannabinol (THC), and any combination thereof.

In some embodiments, the one or more components comprise an aerosol forming agent, selected from the group consisting of a polyhydric alcohol, a sorbitan ester, a fatty acid, a wax, a terpene, and any combination thereof. Certain examples of aerosol forming agents include aerosol forming agents selected from the group consisting of glycerol, propylene glycol, 1,3-propanediol, diethylene glycol, triethylene glycol, sorbitan monolaurate, sorbitan monostearate (Span 60), sorbitan monooleate (Span 20), sorbitan tristearate (Span 65), butyric acid, propionic acid, valeric acid, oleic acid, linoleic acid, stearic acid, myristic acid, palmitic acid, monolaurin, glycerol monostearate, triolein, tripalmitin, tristearate, glycerol tributyrate, glycerol trihexanoate, carnauba wax, beeswax, candellila, limonene, pinene, farnesene, myrcene, geraniol, fennel, cembrene, and any combination thereof.

In some embodiments, the one or more components comprise a flavorant, a filler, an aerosol-forming agent, or any combination thereof, and wherein the incorporating comprises incorporating the component-containing, cross-linked alginate structure as a substrate within a consumable portion of a non-combustible aerosol delivery device.

In some embodiments, the divalent or trivalent cations are selected from the group consisting of calcium (Ca²⁺), barium (Ba²⁺), magnesium (Mg²⁺), strontium (Sr²⁺) iron (Fe²⁺), aluminum (Al³⁺), and combinations thereof. In some embodiments, the contacting step comprises depositing the mixture into a solution comprising the divalent or trivalent cation. A speed of depositing the mixture into the solution is, in some embodiments, controlled to form discrete shapes. The method can, in some embodiments, further comprise casting a sheet of the mixture, and wherein the contacting step comprises contacting the sheet with a solution comprising the divalent or trivalent cations. In certain embodiments, the method further comprises cutting or shredding the component-containing, cross-linked alginate structure to provide strips.

In some embodiments, the contacting step comprises bringing the mixture into contact with an outer surface of a pre-formed structure, wherein the pre-formed structure comprises the divalent or trivalent cation. In some such embodiments, the pre-formed structure is a bead and the mixture is in the form of a coating on the outer surface of the bead. As such, in some embodiments, a component-containing cross-linked alginate structure is provided, in the form of a coating on the outer surface of a bead

In certain embodiments, the component-containing, cross-linked alginate structure is in the form of a sheet, a strip, a bead, or a corkscrew-shaped noodle.

In certain embodiments, the method provided herein further comprises incorporating the component-containing, cross-linked alginate structure within a consumable product. Such consumable products can, in some embodiments, be selected from the group consisting of a product configured for combustible aerosol delivery, a product configured for non-combustible aerosol delivery, and a product configured for aerosol-free delivery.

In some embodiments, a consumable product is provided, selected from the group consisting of an aerosol delivery product, an oral product, and a conventional smoking article, comprising a component-containing cross-linked alginate structure as provided herein.

One example of a consumable product is a consumable product in the form of an oral product, wherein the oral product is a pouched product comprising a pouch at least partially configured with a composition configured for oral use, and wherein the pouch comprises the component-containing cross-linked alginate structure. In some embodiments, the consumable product is completely ingestible after use.

Another example of a consumable product is a product in the form of product configured for non-combustible aerosol delivery, and wherein the component-containing cross-linked alginate structure is a substrate thereof.

A further example of a consumable product is an aerosol generating component comprising a substrate carrying at least one aerosol forming material, the substrate comprising a component-containing cross-linked alginate structure as provided herein. In some such embodiments, the component-containing cross-linked alginate structure further comprises a flavorant and a filler. The aerosol forming material, in some embodiments, glycerin and the filler comprises cellulose-based wood pulp. In certain embodiments, the component-containing cross-linked alginate structure further comprises a nicotine component. The substrate can be, for example, in particulate form, shredded form, film form, paper process sheet form, cast sheet form, bead form, granular rod form, or extrudate form. In some embodiments, the substrate is formed into a substantially cylindrical shape.

The disclosure further provides an aerosol delivery device, comprising: an aerosol generating component as provided herein; a heat source configured to heat the substrate carrying the one or more aerosol forming materials to form an aerosol; and an aerosol pathway extending from the aerosol generating component to a mouth-end of the aerosol delivery device.

The present disclosure includes, without limitation, the following embodiments:

Embodiment 1: A method for providing a composition with a releasable entrapment of one or more components in a component-containing, cross-linked alginate structure, comprising: mixing the one or more components and alginate in water to give a mixture; contacting the mixture with a divalent or trivalent cation to crosslink the alginate, thereby trapping the one or more components within a cross-linked matrix; and removing at least a portion of the water from the cross-linked matrix to give a component-containing alginate structure.

Embodiment 2: The method according to Embodiment 1, wherein the one or more components are selected from the group consisting of flavorants, sweeteners, aerosol-forming agents, humectants, fillers, preservatives, tobacco materials, and combinations thereof.

Embodiment 3: The method according to any one of Embodiments 1-2, wherein the one or more components comprise a flavorant selected from the group consisting of alcohols, aldehydes, aromatic hydrocarbons, ketones, esters, terpenes, terpenoids, trigeminal sensates, and combinations thereof.

Embodiment 4: The method according to any one of Embodiments 1-3, wherein the flavorant is selected from the group consisting of vanillin, ethyl vanillin, p-anisaldehyde, hexanal, furfural, isovaleraldehyde, cuminaldehyde, benzaldehyde, citronellal, 1-hydroxy-2-propanone, 2-hydroxy-3-methyl-2-cyclopentenone-1-one, allyl hexanoate, ethyl heptanoate, ethyl hexanoate, isoamyl acetate, 3-methylbutyl acetate, sabinene, limonene, gamma-terpinene, beta-farnesene, nerolidol, thujone, myrcene, geraniol, nerol, citronellol, linalool, eucalyptol, and combinations thereof.

Embodiment 5: The method according to any one of Embodiments 1-4, wherein the one or more components comprise a flavorant selected from cream, tea, coffee, fruit, maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, Yerba santa, Bacopa monniera, Gingko biloba, Withania somnifera, cinnamon, sandalwood, jasmine, cascarilla, cocoa, licorice, and combinations thereof.

Embodiment 6: The method according to any one of Embodiments 1-5, wherein the one or more components comprise a plant extract.

Embodiment 7: The method according to any one of Embodiments 1-6, wherein the plant extract is a tobacco extract.

Embodiment 8: The method according to any one of Embodiments 1-7, wherein the one or more components comprise an active ingredient, selected from the group consisting of a nicotine component, a botanical/herbal ingredient, a stimulant, an amino acid, a vitamin, an antioxidant, a cannabinoid, a cannabimimetic, a terpene, a pharmaceutical ingredient, and any combination thereof.

Embodiment 9: The method according to any one of Embodiments 1-8, wherein the active ingredient is selected from the group consisting of hemp, guarana, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, maca, tisanes, lemon balm, ginseng, star anise, caffeine, theacrine, theobromine, theophylline, GABA, theanine, taurine, Vitamin B6, Vitamin B12, Vitamin E, Vitamin C, cannabidiol (CBD), tetrahydrocannabinol (THC), and any combination thereof.

Embodiment 10: The method according to any one of Embodiments 1-9, wherein the one or more components comprise an aerosol forming agent, selected from the group consisting of a polyhydric alcohol, a sorbitan ester, a fatty acid, a wax, a terpene, and any combination thereof.

Embodiment 11: The method according to any one of Embodiments 1-10, wherein the aerosol forming agent is selected from the group consisting of glycerol, propylene glycol, 1,3-propanediol, diethylene glycol, triethylene glycol, sorbitan monolaurate, sorbitan monostearate (Span 60), sorbitan monooleate (Span 20), sorbitan tristearate (Span 65), butyric acid, propionic acid, valeric acid, oleic acid, linoleic acid, stearic acid, myristic acid, palmitic acid, monolaurin, glycerol monostearate, triolein, tripalmitin, tristearate, glycerol tributyrate, glycerol trihexanoate, carnauba wax, beeswax, candellila, limonene, pinene, farnesene, myrcene, geraniol, fennel, cembrene, and any combination thereof.

Embodiment 12: The method according to any one of Embodiments 1-11, wherein the one or more components comprise a flavorant, a filler, an aerosol-forming agent, or any combination thereof, and wherein the incorporating comprises incorporating the component-containing, cross-linked alginate structure as a substrate within a consumable portion of a non-combustible aerosol delivery device.

Embodiment 13: The method according to any one of Embodiments 1-12, wherein the divalent or trivalent cations are selected from the group consisting of calcium (Ca²⁺), barium (Ba²⁺), magnesium (Mg²⁺), strontium (Sr²⁺) iron (Fe²⁺), aluminum (Al³⁺), and combinations thereof.

Embodiment 14: The method according to any one of Embodiments 1-13, wherein the contacting step comprises depositing the mixture into a solution comprising the divalent or trivalent cation.

Embodiment 15: The method according to any one of Embodiments 1-14, wherein a speed of depositing the mixture into the solution is controlled to form discrete shapes.

Embodiment 16: The method according to any one of Embodiments 1-15, further comprising casting a sheet of the mixture, and wherein the contacting step comprises contacting the sheet with a solution comprising the divalent or trivalent cations.

Embodiment 17: The method according to any one of Embodiments 1-16, further comprising cutting or shredding the component-containing, cross-linked alginate structure to provide strips.

Embodiment 18: The method according to any one of Embodiments 1-17, wherein the contacting step comprises bringing the mixture into contact with an outer surface of a pre-formed structure, wherein the pre-formed structure comprises the divalent or trivalent cation.

Embodiment 19 The method according to any one of Embodiments 1-18, wherein the pre-formed structure is a bead and the mixture is in the form of a coating on the outer surface of the bead.

Embodiment 20: The method according to any one of Embodiments 1-19, wherein the component-containing, cross-linked alginate structure is in the form of a sheet, a strip, a bead, or a corkscrew-shaped noodle.

Embodiment 21: The method according to any one of Embodiments 1-20, further comprising incorporating the component-containing, cross-linked alginate structure within a consumable product.

Embodiment 22: The method according to any one of Embodiments 1-21, wherein the consumable product is selected from the group consisting of a product configured for combustible aerosol delivery, a product configured for non-combustible aerosol delivery, or a product configured for aerosol-free delivery.

Embodiment 23: A component-containing cross-linked alginate structure, prepared according to the method according to any one of Embodiments 1-22.

Embodiment 24: A component-containing cross-linked alginate structure, comprising one or more components entrapped within a cross-linked alginate matrix.

Embodiment 25: The component-containing cross-linked alginate structure according to any one of Embodiments 23-24, wherein the one or more components are selected from the group consisting of flavorants, sweeteners, aerosol-forming agents, humectants, fillers, preservatives, tobacco materials, and combinations thereof.

Embodiment 26: The component-containing cross-linked alginate structure according to any one of Embodiments 23-25, wherein the one or more components comprise a flavorant selected from the group consisting of alcohols, aldehydes, aromatic hydrocarbons, ketones, esters, terpenes, terpenoids, trigeminal sensates, and combinations thereof.

Embodiment 27: The component-containing cross-linked alginate structure of according to any one of Embodiments 23-26, wherein the flavorant is selected from the group consisting of vanillin, ethyl vanillin, p-anisaldehyde, hexanal, furfural, isovaleraldehyde, cuminaldehyde, benzaldehyde, citronellal, 1-hydroxy-2-propanone, 2-hydroxy-3-methyl-2-cyclopentenone-1-one, allyl hexanoate, ethyl heptanoate, ethyl hexanoate, isoamyl acetate, 3-methylbutyl acetate, sabinene, limonene, gamma-terpinene, beta-farnesene, nerolidol, thujone, myrcene, geraniol, nerol, citronellol, linalool, eucalyptol, and combinations thereof.

Embodiment 28: The component-containing cross-linked alginate structure according to any one of Embodiments 23-27, wherein the one or more components comprise a flavorant selected from cream, tea, coffee, fruit, maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, Yerba santa, Bacopa monniera, Gingko biloba, Withania somnifera, cinnamon, sandalwood, jasmine, cascarilla, cocoa, licorice, and combinations thereof.

Embodiment 29: The component-containing cross-linked alginate structure according to any one of Embodiments 23-28, wherein the one or more components comprise a plant extract.

Embodiment 30: The component-containing cross-linked alginate structure according to any one of Embodiments 23-29, wherein the plant extract is a tobacco extract.

Embodiment 31: The component-containing cross-linked alginate structure according to any one of Embodiments 23-30, wherein the one or more components comprise an active ingredient, selected from the group consisting of a nicotine component, a botanical/herbal ingredient, a stimulant, an amino acid, a vitamin, an antioxidant, a cannabinoid, a cannabimimetic, a terpene, a pharmaceutical ingredient, and any combination thereof.

Embodiment 32: The component-containing cross-linked alginate structure according to any one of Embodiments 23-31, wherein the active ingredient is selected from the group consisting of hemp, guarana, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, maca, tisanes, lemon balm, ginseng, star anise, caffeine, theacrine, theobromine, theophylline, GABA, theanine, taurine, Vitamin B6, Vitamin B12, Vitamin E, Vitamin C, cannabidiol (CBD), tetrahydrocannabinol (THC), and any combination thereof.

Embodiment 33: The component-containing cross-linked alginate structure according to any one of Embodiments 23-32, wherein the one or more components comprise an aerosol forming agent, selected from the group consisting of a polyhydric alcohol, a sorbitan ester, a fatty acid, a wax, a terpene, and any combination thereof.

Embodiment 34: The component-containing cross-linked alginate structure according to any one of Embodiments 23-33, wherein the aerosol forming agent is selected from the group consisting of glycerol, propylene glycol, 1,3-propanediol, diethylene glycol, triethylene glycol, sorbitan monolaurate, sorbitan monostearate (Span 60), sorbitan monooleate (Span 20), sorbitan tristearate (Span 65), butyric acid, propionic acid, valeric acid, oleic acid, linoleic acid, stearic acid, myristic acid, palmitic acid, monolaurin, glycerol monostearate, triolein, tripalmitin, tristearate, glycerol tributyrate, glycerol trihexanoate, carnauba wax, beeswax, candellila, limonene, pinene, farnesene, myrcene, geraniol, fennel, cembrene, and any combination thereof.

Embodiment 35: The component-containing cross-linked alginate structure according to any one of Embodiments 23-34, wherein the one or more components comprise a flavorant, a filler, an aerosol-forming agent, or any combination thereof, and wherein the incorporating comprises incorporating the component-containing, cross-linked alginate structure as a substrate within a consumable portion of a non-combustible aerosol delivery device.

Embodiment 36: The component-containing cross-linked alginate structure according to any one of Embodiments 23-35, in the form of a coating on the outer surface of a bead.

Embodiment 37: The component-containing cross-linked alginate structure according to any one of Embodiments 23-36, in the form of a sheet, a strip, a bead, or a corkscrew-shaped noodle.

Embodiment 38: A consumable product selected from the group consisting of an aerosol delivery product, an oral product, and a conventional smoking article, comprising the component-containing cross-linked alginate structure according to any one of Embodiments 23-37.

Embodiment 39: The consumable product according to Embodiment 38, in the form of an oral product, wherein the oral product is a pouched product comprising a pouch at least partially configured with a composition configured for oral use, and wherein the pouch comprises the component-containing cross-linked alginate structure.

Embodiment 40: The consumable product according to any one of Embodiments 38-39, wherein the consumable product is completely ingestible after use.

Embodiment 41: The consumable product according to any one of Embodiments 38-40, in the form of a product configured for non-combustible aerosol delivery, and wherein the component-containing cross-linked alginate structure is a substrate thereof.

Embodiment 42: An aerosol generating component comprising a substrate carrying at least one aerosol forming material, the substrate comprising the component-containing cross-linked alginate structure according to any one of Embodiments 23-37.

Embodiment 43: The aerosol generating component according to Embodiment 42, wherein the component-containing cross-linked alginate structure further comprises a flavorant and a filler.

Embodiment 44: The aerosol generating component according to any one of Embodiments 42-43, wherein the aerosol forming material comprises glycerin and the filler comprises cellulose-based wood pulp.

Embodiment 45: The aerosol generating component according to any one of Embodiments 42-44, wherein the component-containing cross-linked alginate structure further comprises a nicotine component.

Embodiment 46: The aerosol generating component according to any one of Embodiments 42-45, wherein the substrate is in particulate form, shredded form, film form, paper process sheet form, cast sheet form, bead form, granular rod form, or extrudate form.

Embodiment 47: The aerosol generating component according to any one of Embodiments 42-46, wherein the substrate is formed into a substantially cylindrical shape.

Embodiment 48: An aerosol delivery device, comprising: the aerosol generating component according to any one of Embodiments 42-47; a heat source configured to heat the substrate carrying the one or more aerosol forming materials to form an aerosol; and an aerosol pathway extending from the aerosol generating component to a mouth-end of the aerosol delivery device.

These and other features, aspects, and advantages of the disclosure will be apparent from a reading of the following detailed description together with the accompanying drawings, which are briefly described below. The invention includes any combination of two, three, four, or more of the above-noted embodiments as well as combinations of any two, three, four, or more features or elements set forth in this disclosure, regardless of whether such features or elements are expressly combined in a specific embodiment description herein. This disclosure is intended to be read holistically such that any separable features or elements of the disclosed invention, in any of its various aspects and embodiments, should be viewed as intended to be combinable unless the context clearly dictates otherwise. Other aspects and advantages of the present disclosure will become apparent from the following.

### BRIEF DESCRIPTION OF THE DRAWINGS

Having thus described aspects of the disclosure in the foregoing general terms, reference will now be made to the accompanying drawings, which are not necessarily drawn to scale. The drawings are examples only, and should not be construed as limiting the disclosure.

FIG. 1 provides an overview of certain method steps associated with an embodiment of the method outlined herein (“Process A”) for the production of a component-containing, alginate-based substrate;

FIGS. 2A-2H illustrate various non-limiting forms of the component-containing, alginate-based substrate;

FIG. 3 illustrates a further form of a component-containing, alginate-based substrate (as the shell of a core-shell structure);

FIG. 4 provides an overview of certain method steps associated with an embodiment of the method outlined herein (“Process B”) for the production of a component-containing, alginate-based substrate;

FIG. 5 provides a perspective view of an aerosol delivery device comprising a control body and an aerosol generating component, wherein the generating component and the control body are coupled to one another, according to an example embodiment of the present disclosure;

FIG. 6 illustrates a perspective view of the aerosol delivery device of FIG. 5 wherein the aerosol generating component and the control body are decoupled from one another, according to an example embodiment of the present disclosure;

FIG. 7 illustrates a perspective schematic view of an aerosol generating component, according to an example embodiment of the disclosure;

FIG. 8 illustrates a schematic cross-section drawing of a substrate portion of an aerosol generating component, according to an example embodiment of the present disclosure;

FIG. 9 illustrates a perspective view of an aerosol delivery device comprising a control body and an aerosol generating component, wherein the generating component and the control body are coupled to one another, according to one or more embodiments of the present disclosure;

FIG. 10 illustrates a perspective view of the aerosol delivery device of FIG. 9, wherein the aerosol generating component and the control body are decoupled from one another, according to one or more embodiments of the present disclosure;

FIG. 11 is a perspective view of a pouched product embodiment according to an example embodiment of the present disclosure including a pouch or fleece, the pouch or fleece comprising an embodiment of the disclosed component-containing, alginate-based substrate, which is at least partially filled with a composition configured for oral use; and

FIG. 12 is an exploded perspective view of a conventional smoking article having the form of a cigarette, showing the smokable material, the wrapping material components, and the filter element of the cigarette.

### DETAILED DESCRIPTION

The present disclosure will now be described more fully hereinafter with reference to example embodiments thereof. These example embodiments are described so that this disclosure will be thorough and complete, and will fully convey the scope of the disclosure to those skilled in the art. Indeed, the disclosure may be embodied in many different forms and should not be construed as limited to the embodiments set forth herein; rather, these embodiments are provided so that this disclosure will satisfy applicable legal requirements. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water). Reference to percent is intended to mean percent by weight unless otherwise indicated.

As described hereinafter, example embodiments of the present disclosure relate to components (e.g., including volatile components) incorporated/entrapped within an alginate matrix, i.e., component-containing, alginate-based substrates, and to methods of providing and using such alginate-based substrates. Further example embodiments of the present disclosure relate to products incorporating such component-containing, alginate-based substrates including, but not limited to, an aerosol delivery device comprising the component-containing, alginate-based substrate as disclosed herein (e.g., within the substrate thereof); a heat source configured to heat aerosol forming materials impregnated in the substrate portion to form an aerosol (and release one or more components from the component-containing, alginate-based substrate); and an aerosol pathway extending from the aerosol generating component to a mouth-end of the aerosol delivery device.

By “entrapped” or “containing” as used herein is meant that the component (or components) is within the alginate matrix. The entrapment/containing of the one or more components typically comprises physical containment, i.e., the component (or components) is/are physically held within the alginate matrix until released. Typically, this physical containment is provided by cross-linking within the alginate matrix, although the containment is not limited thereto. The entrapment provided herein does not exclude the formation of ionic or covalent bonds between a component (or components) and the alginate matrix. Typically, the entrapped components are sufficiently contained so as to remain within the alginate matrix for a certain period of time and/or under certain conditions. For example, the entrapped components typically stay sufficiently contained within the alginate matrix to allow for their inclusion within a desired product (e.g., including, but not limited to, an aerosol delivery device) without being substantially released from the alginate matrix. The entrapped components are generally only temporarily entrapped and may be released from the alginate matrix upon exposure to certain stimuli (e.g., heat). As such, for example, component(s) desirably released to the user of an aerosol delivery device can be entrapped during production and storage of the device and released from the alginate matrix during use (when the alginate matrix is subjected to heat), maximizing the amount of such a component (or components) that is retained during production and storage of the product and then provided to the user.

### Method of Incorporation

### Process A

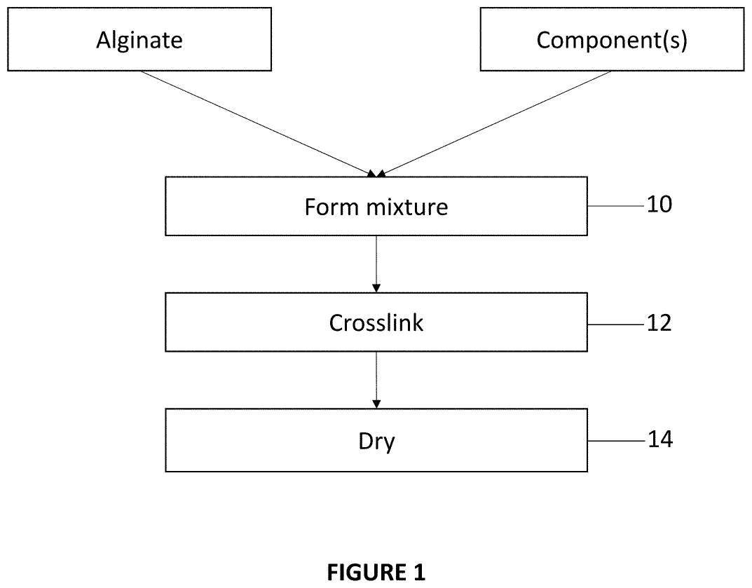

Various components can be temporarily entrapped within an alginate polymer matrix according to the present disclosure, giving a component-containing, alginate-based substrate, as will be described further herein. An example process (referred to herein as “Process A”) is provided in FIG. 1, wherein step 10 comprises forming a mixture from alginate and a component to be encapsulated therein; step 12 comprises cross-linking the alginate, and step 14 comprises drying the resulting material to give a component-containing, alginate-based substrate.

### Mixing—Step 10

In some embodiments, step 10 comprises mixing one or more components with alginate. Typically, although not limited thereto, the one or more components and the alginate are mixed in a solvent, e.g., water. The resulting mixture can be in various forms, e.g., depending on the form of the components, e.g., in the form of a slurry or a solution. The form of the mixture can vary based, e.g., on the solubility of the component(s) other than the alginate in the liquid used in mixing step 10 and the exact type of alginate used.

In some embodiments, step 10 comprises forming a slurry. “Slurry” as used herein is understood to have its general definition as known in the art, e.g., a mixture of solids suspended in a liquid. The slurry can be of varying concentrations and its viscosity can be adjusted, e.g., by selection of components, by the addition of or removal of liquid (and/or by the addition of or removal of solids). The concentration of the slurry may be specifically optimized based on its “flowability,” as for subsequent processing, the slurry is advantageously thick enough to hold its shape to be formed into desirable forms, but not too thick to allow for it to flow and be formed into such desired forms. Typically, in such embodiments, the alginate is substantially dissolved in the liquid; the component(s) to be entrapped therein can also be dissolved or can be suspended/dispersed within the slurry (e.g., forming the solid component of the slurry).

A slurry may be, in some embodiments, defined by its solids fraction/percent solids by mass (Φₛₗ). The solids fraction for a given slurry can be calculated by the following formula:

Φₛₗ=mass (solids)/mass (slurry).

Where the mixture is in the form of a solution, the concentration of the solution can vary. As known in the art, a solution can be defined, e.g., by its mass percent, volume percent, or molarity.

Advantageously, the mixture provided in step 10 is substantially homogeneous (including completely homogeneous). As such, the mixing step is typically conducted for a period of time to sufficiently mix the components thereof to provide such a mixture (e.g., slurry or solution). The exact time can vary depending, e.g., on the solids fraction of the slurry (as slurries with higher solids fractions may take more time to thoroughly mix than those with lower solids fractions) or concentration of the solution. Such mixing is generally conducted at room temperature; however, the conditions are not intended to be limiting (the mixing can alternatively be conducted at reduced or elevated temperatures). The rate of mixing is not particularly limited; again, it is typically a sufficient rate to ensure substantial homogeneity (or complete homogeneity) in a reasonable period of time. Mixing can be conducted, e.g., by mechanical stirring/agitation (which can be done by hand or via equipment for such purposes, such as mixers, blenders, and the like).

Alginate refers to a linear unbranched anionic heteropolysaccharide as known in the art. Alginates consist of different amounts of linear copolymers of β-(1-4) linked D-mannuronic acid and β-(1-4) linked L-guluronic acid (often referred to, respectively, as “M” and “G” residues). Alginates are typically block copolymers, with blocks of consecutive residues (e.g., GGGGG and MMMMMM) and regions of alternating residues GMGMGMGMG. The molecular weights of alginates can vary widely, e.g., between about 32,000 and 400,000, with higher molecular weight alginates typically providing a more viscous slurry and lower molecular weight alginates providing a less viscous slurry.

Alginates are typically natural polymers and can, in some embodiments, be derived from seaweeds. Certain commercially available alginates are extracted from brown algae (Phaeophycae), including (but not limited to) Laminaria hyperborean, Laminaria digitate, Laminaria japonica, Ascophyllum nodosum, and Macrocystis pyrifera. Alginates from different sources differ in M and G residue content and block lengths. Alginates can also be in the form of a synthetic polymer (provided via bacterial biosynthesis, e.g., produced from Azotobacter or Pseudomonas). Alginates are typically in the form of a sodium, calcium, or manganese salt (but can also be in the form of other alginate salts). In preferred embodiments herein, the alginate is employed in a water-soluble form.

The “component(s)” mixed with the alginate in step 10 can be in the solid or the liquid phase of the mixture. Such components vary widely and can depend on the desired attributes of the final product. For example, in some embodiments, the component(s) can include one or more of a flavorant, a tobacco material, a botanical material, an active ingredient, a sweetener, an aerosol forming agent, a preservative, and/or a filler. In some embodiments, the component(s) comprise a volatile component, such that containment within the alginate-based material provided according to the disclosed method serves to protect the component(s) from premature volatilization prior to use (e.g., during preparation or storage of the product into which it is incorporated).

In some embodiments, the component comprises a flavorant (also referred to as a “flavor material,” “flavor,” “flavoring,” or “flavoring agent”). A wide range of flavorants are known and can be suitably encapsulated via this method. As used herein, reference to a “flavorant” refers to compounds or components that can be aerosolized and delivered to a user and which impart a sensory experience in terms of taste and/or aroma. Examples of sensory characteristics that can be modified by the flavor material include, taste, mouth feel, moistness, coolness/heat, and/or fragrance/aroma.

Flavorants can be provided from tobacco or from sources other than tobacco, can be natural or synthetic, and the character of these flavors can be described as, without limitation, fresh, sweet, herbal, confectionary, floral, fruity or spice. Such flavoring agents can, in some embodiments, be employed as concentrates or flavor packages. Some examples of flavorants include, but are not limited to, vanillin, ethyl vanillin, cream, tea, coffee, fruit (e.g., apple, cherry, strawberry, peach and citrus flavors, including lime and lemon), maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, Yerba santa, Bacopa monniera, Gingko biloba, Withania somnifera, cinnamon, sandalwood, jasmine, cascarilla, cocoa, licorice, and flavorings and flavor packages of the type and character traditionally used for the flavoring of cigarette, cigar, and pipe tobaccos. Some examples of plant-derived compositions that may be suitable are disclosed in U.S. Pat. No. 9,107,453 and U.S. Pat. App. Pub. No. 2012/0152265 both to Dube et al., the disclosures of which are incorporated herein by reference in their entireties. The selection of such flavoring components is variable based upon factors such as the sensory characteristics that are desired for the product the component is designed for incorporation within, their affinity for the substrate material (and suitability for forming a slurry with alginate), their solubility, and other physiochemical properties. The present disclosure is intended to encompass any such further components that are readily apparent to those skilled in the art of tobacco and tobacco-related or tobacco-derived products. See, e.g., Gutcho, Tobacco Flavoring Substances and Methods, Noyes Data Corp. (1972) and Leffingwell et al., Tobacco Flavoring for Smoking Products (1972), the disclosures of which are incorporated herein by reference in their entireties. It should be noted that reference to a flavorant should not be limited to any single flavorant as described above, and may, in fact, represent a combination of one or more flavorants. Additional flavorants, flavoring agents, additives, and other possible enhancing constituents are described in U.S. Pat. App. Pub. No. 2019/0082735 to Phillips et al., which is incorporated herein by reference in its entirety.

In some embodiments, flavorants are plant extracts. Extracts selected for use in certain embodiments of the disclosed methods and materials can be derived from a variety of species, using a variety of techniques that produce extract in a variety of usable forms, such as a tobacco extract or similar flavor being derived from a plant of the Nicotiana species. As used herein, the term “tobacco extract” means components separated from, removed from, or derived from, tobacco using tobacco extraction processing conditions and techniques. Purified extracts of tobacco or other botanicals specifically can be used. Typically, tobacco extracts are obtained using solvents, such as solvents having an aqueous nature (e.g., water) or organic solvents (e.g., alcohols, such as ethanol or alkanes, such as hexane). As such, extracted tobacco components are removed from tobacco and separated from the unextracted tobacco components; and for extracted tobacco components that are present within a solvent, (i) the solvent can be removed from the extracted tobacco components, or (ii) the mixture of extracted tobacco components and solvent can be used as such. Examples of types of tobacco extracts, tobacco essences, solvents, tobacco extraction processing conditions and techniques, and tobacco extract collection and isolation procedures, are set forth in Australia Pat. No. 276,250 to Schachner; U.S. Pat. No. 2,805,669 to Meriro; U.S. Pat. No. 3,316,919 to Green et al.; U.S. Pat. No. 3,398,754 to Tughan; U.S. Pat. No. 3,424,171 to Rooker; U.S. Pat. No. 3,476,118 to Luttich; U.S. Pat. No. 4,150,677 to Osborne; U.S. Pat. No. 4,131,117 to Kite; U.S. Pat. No. 4,506,682 to Muller; U.S. Pat. No. 4,986,286 to Roberts et al.; U.S. Pat. No. 5,005,593 to Fagg; U.S. Pat. No. 5,065,775 to Fagg; U.S. Pat. No. 5,060,669 to White et al.; U.S. Pat. No. 5,074,319 to White et al.; U.S. Pat. No. 5,099,862 to White et al.; U.S. Pat. No. 5,121,757 to White et al.; U.S. Pat. No. 5,131,415 to Munoz et al.; U.S. Pat. No. 5,230,354 to Smith et al.; U.S. Pat. No. 5,235,992 to Sensabaugh; U.S. Pat. No. 5,243,999 to Smith; U.S. Pat. No. 5,301,694 to Raymond; U.S. Pat. No. 5,318,050 to Gonzalez-Parra et al.; U.S. Pat. No. 5,435,325 to Clapp et al.; and U.S. Pat. No. 5,445,169 to Brinkley et al., which are incorporated herein by reference in their entireties. Where a tobacco extract is included as a component, it can be in an amount up to about 5 percent by weight, up to about 3 percent by weight, up to about 2 percent by weight, or up to about 1 percent by weight, e.g., about 0.1 to about 5 percent by weight based on the alginate mixture.

Certain flavorants that are beneficially applicable in the present disclosure are volatile flavor components. As used herein, “volatile” refers to a chemical substance that forms a vapor readily at ambient temperatures (i.e., a chemical substance that has a high vapor pressure at a given temperature relative to a non-volatile substance). Typically, a volatile flavor compound has a molecular weight below about 400 Da and often includes at least one carbon-carbon double bond, carbon-oxygen double bond, or both. In some embodiments, volatile flavor components that are advantageously incorporated within an alginate substrate as provided herein comprise one or more alcohols, aldehydes, aromatic hydrocarbons, ketones, esters, terpenes, terpenoids, trigeminal sensates. Non-limiting examples of aldehydes include vanillin, ethyl vanillin, p-anisaldehyde, hexanal, furfural, isovaleraldehyde, cuminaldehyde, benzaldehyde, and citronellal. Non-limiting examples of ketones include 1-hydroxy-2-propanone and 2-hydroxy-3-methyl-2-cyclopentenone-1-one. Non-limiting examples of esters include allyl hexanoate, ethyl heptanoate, ethyl hexanoate, isoamyl acetate, and 3-methylbutyl acetate. Non-limiting examples of terpenes include sabinene, limonene, gamma-terpinene, beta-farnesene, nerolidol, thujone, myrcene, geraniol, nerol, citronellol, linalool, and eucalyptol.

In some embodiments, the flavorant comprises menthol, spearmint and/or peppermint. In some embodiments, the flavorant comprises flavor components of cucumber, blueberry, citrus fruits and/or redberry. In some embodiments, the flavorant comprises eugenol. In some embodiments, the flavorant comprises flavor components extracted from tobacco. In some embodiments, the flavorant comprises flavor components extracted from cannabis.

In some embodiments, the flavorant may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to, eucolyptol or WS-3. Flavorants, including extracts, may be provided in various forms, e.g., a liquid form or a substantially solid (e.g., powder or pellet-type) form. The flavorant may also, in some embodiments, be in encapsulated form. The encapsulated form may include a wall or barrier structure defining an inner region or payload that contains the flavor material. Use of additives in microencapsulated form can improve storage stability of the product, particularly the stability of the sensory profile of the product, and protect certain additives from degradation over time. Microencapsulation can also insulate the user from undesirable sensory characteristics associated with the encapsulated ingredient, such as certain fillers, or provide a milder sensory experience by extending the release of certain flavorants over time. A representative microcapsule embodiment has an outer cover, shell, or coating that envelopes a liquid or solid core region, and in certain embodiments, the microcapsule can have a generally spherical shape. By encapsulating an additive within the core region of a microcapsule, the ability of the additive to interact with other components of the product is reduced or eliminated, which can enhance the storage stability of the resulting product. The core region, which typically releases the flavorant payload when the outer shell undergoes some type of physical destruction, breakage, or other loss of physical integrity (e.g., through dispersion, softening, crushing, application of pressure, or the like), thereby provides for altering the sensory properties of the product into which it is incorporated. Thus, in many embodiments, the outer shell of the microcapsules is designed to rupture during use or is water soluble under conditions of normal use.

Examples of manners and methods for providing encapsulated materials, such as microencapsulated flavoring agents, are set forth in Gutcho, Microcapsules and Microencapsulation Techniques (1976) and Gutcho, Microcapsules and Other Capsules Advances Since 1975 (1979), which are incorporated herein by reference. Certain types of microcapsules can have diameters of less than 100 microns, and often can have outer shells that are gelatin based, cyclodextrin based, or the like. Microcapsules have been commercially available, and examples of types of microcapsule technologies are of that type set forth in Kondo, Microcapsule Processing and Technology (1979); Iwamoto et al., AAPS Pharm. Sci. Tech. 2002 3(3): article 25; and U.S. Pat. No. 3,550,598 to McGiumphy and U.S. Pat. No. 6,117,455 to Takada et al. Flavorants may also, in some embodiments, be provided in selectively crushable capsules which are designed such that the user may control if, when, and how much flavor is consumed from the product.

The quantity of flavorant present within the alginate matrix of the present disclosure may vary. When the component-containing, alginate-based substrates provided herein comprise one or more flavorants, the content of such flavorants is generally up to about 40% by dry weight of the final substrate, e.g., in some embodiments, about 40% or less, about 30% or less, or about 20% or less by dry weight of the substrate. Such components (and others provided herein, where relevant) are conveniently calculated on a dry weight basis with the final moisture of the product since moisture can vary in the wet mixture. For example, a flavorant may be present in a quantity of from about 0.1%, about 0.5%, about 1%, or about 5%, to about 10%, about 20%, about 30%, or about 40% by dry weight of the final product. This amount is generally provided as a dry weight, as the amount of water in the mixture and in the final substrate can vary. Amounts of flavorants to be provided within the mixture (i.e., slurry) provided by step 10 can be determined accordingly to obtain the desired amount of flavorant in the final component-containing, alginate-based substrate.

Another component that can be suitably incorporated within an alginate matrix according to certain methods provided herein is a tobacco material. For example, in some embodiments, the component provided in the slurry can comprise tobacco material, e.g., in particulate form. Where tobacco (in the form of an extract, as referenced above, or an alternative form, e.g., particulate form) is incorporated as a component in mixing step 10, it is noted that the tobacco can be tobacco of various species, type, and form. Generally, tobacco material, where present, is obtained from for a harvested plant of the Nicotiana species. Example Nicotiana species include N. tabacum, N. rustica, N. alata, N. arentsii, N. excelsior, N. forgetiana, N. glauca, N. glutinosa, N. gossei, N. kawakamii, N. knightiana, N. langsdorffi, N. otophora, N. setchelli, N. sylvestris, N. tomentosa, N. tomentosiformis, N. undulata, N. x sanderae, N. africana, N. amplexicaulis, N. benavidesii, N. bonariensis, N. debneyi, N. longiflora, N. maritina, N. megalosiphon, N. occidentalis, N. paniculata, N. plumbaginifolia, N. raimondii, N. rosulata, N. simulans, N. stocktonii, N. suaveolens, N. umbratica, N. velutina, N. wigandioides, N. acaulis, N. acuminata, N. attenuata, N. benthamiana, N. cavicola, N. clevelandii, N. cordifolia, N. corymbosa, N. fragrans, N. goodspeedii, N. linearis, N. miersii, N. nudicaulis, N. obtusifolia, N. occidentalis subsp. Hersperis, N. pauciflora, N. petunioides, N. quadrivalvis, N. repanda, N. rotundifolia, N. solanifolia, and N. spegazzinii. Various representative other types of plants from the Nicotiana species are set forth in Goodspeed, The Genus Nicotiana, (Chonica Botanica) (1954); U.S. Pat. No. 4,660,577 to Sensabaugh, Jr. et al.; U.S. Pat. No. 5,387,416 to White et al., U.S. Pat. No. 7,025,066 to Lawson et al.; U.S. Pat. No. 7,798,153 to Lawrence, Jr. and U.S. Pat. No. 8,186,360 to Marshall et al.; each of which is incorporated herein by reference. Descriptions of various types of tobaccos, growing practices and harvesting practices are set forth in Tobacco Production, Chemistry and Technology, Davis et al. (Eds.) (1999), which is incorporated herein by reference.

Nicotiana species from which suitable tobacco materials can be obtained can be derived using genetic-modification or crossbreeding techniques (e.g., tobacco plants can be genetically engineered or crossbred to increase or decrease production of components, characteristics or attributes). See, for example, the types of genetic modifications of plants set forth in U.S. Pat. No. 5,539,093 to Fitzmaurice et al.; U.S. Pat. No. 5,668,295 to Wahab et al.; U.S. Pat. No. 5,705,624 to Fitzmaurice et al.; U.S. Pat. No. 5,844,119 to Weigl; U.S. Pat. No. 6,730,832 to Dominguez et al.; U.S. Pat. No. 7,173,170 to Liu et al.; U.S. Pat. No. 7,208,659 to Colliver et al. and U.S. Pat. No. 7,230,160 to Benning et al.; US Patent Appl. Pub. No. 2006/0236434 to Conkling et al.; and PCT WO2008/103935 to Nielsen et al. See, also, the types of tobaccos that are set forth in U.S. Pat. No. 4,660,577 to Sensabaugh, Jr. et al.; U.S. Pat. No. 5,387,416 to White et al.; and U.S. Pat. No. 6,730,832 to Dominguez et al., each of which is incorporated herein by reference.

The Nicotiana species can, in some embodiments, be selected for the content of various compounds that are present therein. For example, plants can be selected on the basis that those plants produce relatively high quantities of one or more of the compounds desired to be isolated therefrom. In certain embodiments, plants of the Nicotiana species (e.g., Galpao commun tobacco) are specifically grown for their abundance of leaf surface compounds. Tobacco plants can be grown in greenhouses, growth chambers, or outdoors in fields, or grown hydroponically.

Various parts or portions of the plant of the Nicotiana species can be included within a mixture as disclosed herein. For example, virtually all of the plant (e.g., the whole plant) can be harvested, and employed as such. Alternatively, various parts or pieces of the plant can be harvested or separated for further use after harvest. For example, the flower, leaves, stem, stalk, roots, seeds, and various combinations thereof, can be isolated for use as a component within an alginate matrix as provided herein. In some embodiments, the tobacco material comprises tobacco leaf (lamina). The mixture disclosed herein can include processed tobacco parts or pieces, cured and aged tobacco in essentially natural lamina and/or stem form, a tobacco extract, extracted tobacco pulp (e.g., using water as a solvent), or a mixture of the foregoing (e.g., a mixture that combines extracted tobacco pulp with granulated cured and aged natural tobacco lamina).

In certain embodiments, the tobacco material comprises solid tobacco material selected from the group consisting of lamina and/or stems. Portions of the tobaccos within the mixture may have processed forms, such as processed tobacco stems (e.g., cut-rolled stems, cut-rolled-expanded stems or cut-puffed stems), or volume expanded tobacco (e.g., puffed tobacco, such as dry ice expanded tobacco (DIET)). See, for example, the tobacco expansion processes set forth in U.S. Pat. No. 4,340,073 to de la Burde et al.; U.S. Pat. No. 5,259,403 to Guy et al.; and U.S. Pat. No. 5,908,032 to Poindexter, et al.; and U.S. Pat. No. 7,556,047 to Poindexter, et al., all of which are incorporated by reference. In addition, the d mixture optionally may incorporate tobacco that has been fermented. See, also, the types of tobacco processing techniques set forth in International Patent Application Publication No. WO2005/063060 to Atchley et al., which is incorporated herein by reference.

Tobacco material, where present, is typically used in a form that can be described as particulate (i.e., shredded, ground, granulated, or powder form). The manner by which the tobacco material is provided in a finely divided or powder type of form may vary. Preferably, plant parts or pieces are comminuted, ground or pulverized into a particulate form using equipment and techniques for grinding, milling, or the like. Most preferably, the plant material is relatively dry in form during grinding or milling, using equipment such as hammer mills, cutter heads, air control mills, or the like. For example, tobacco parts or pieces may be ground or milled when the moisture content thereof is less than about 15 weight percent or less than about 5 weight percent. Most preferably, the tobacco material is employed in the form of parts or pieces that have an average particle size between 1.4 millimeters and 400 microns, including those having an average particle size of about 250 microns and below. In some instances, the tobacco particles may be sized to pass through a screen mesh to obtain the particle size range required. If desired, air classification equipment may be used to ensure that small sized tobacco particles of the desired sizes, or range of sizes, may be collected. If desired, differently sized pieces of granulated tobacco may be mixed together.

The manner by which particulate tobacco is provided in a finely divided or powder type of form may vary. Preferably, tobacco parts or pieces are comminuted, ground or pulverized into a powder type of form using equipment and techniques for grinding, milling, or the like. Most preferably, the tobacco is relatively dry in form during grinding or milling, using equipment such as hammer mills, cutter heads, air control mills, or the like. For example, tobacco parts or pieces may be ground or milled when the moisture content thereof is less than about 15 weight percent to less than about 5 weight percent. For example, the tobacco plant or portion thereof can be separated into individual parts or pieces (e.g., the leaves can be removed from the stems, and/or the stems and leaves can be removed from the stalk). The harvested plant or individual parts or pieces can be further subdivided into parts or pieces (e.g., the leaves can be shredded, cut, comminuted, pulverized, milled or ground into pieces or parts that can be characterized as filler-type pieces, granules, particulates or fine powders). The plant, or parts thereof, can be subjected to external forces or pressure (e.g., by being pressed or subjected to roll treatment). When carrying out such processing conditions, the plant or portion thereof can have a moisture content that approximates its natural moisture content (e.g., its moisture content immediately upon harvest), a moisture content achieved by adding moisture to the plant or portion thereof, or a moisture content that results from the drying of the plant or portion thereof. For example, powdered, pulverized, ground or milled pieces of plants or portions thereof can have moisture contents of less than about 25 weight percent, often less than about 20 weight percent, and frequently less than about 15 weight percent.

It is typical for a harvested plant of the Nicotiana species to be subjected to a curing process before inclusion within a mixture with alginate, as provided herein. The tobacco materials optionally incorporated within the mixture for inclusion within products as disclosed herein are those that have been appropriately cured and/or aged. Descriptions of various types of curing processes for various types of tobaccos are set forth in Tobacco Production, Chemistry and Technology, Davis et al. (Eds.) (1999), which is incorporated herein by reference. Examples of techniques and conditions for curing flue-cured tobacco are set forth in Nestor et al., Beitrage Tabakforsch. Int, 20, 467-475 (2003) and U.S. Pat. No. 6,895,974 to Peele, which are incorporated herein by reference. Representative techniques and conditions for air curing tobacco are set forth in U.S. Pat. No. 7,650,892 to Groves et al.; Roton et al., Beitrage Tabakforsch. Int, 21, 305-320 (2005) and Staaf et al., Beitrage Tabakforsch. Int, 21, 321-330 (2005), which are incorporated herein by reference. Certain types of tobaccos can be subjected to alternative types of curing processes, such as fire curing or sun curing. In certain embodiments, tobacco materials that can be employed include flue-cured or Virginia (e.g., K326), burley, sun-cured (e.g., Indian Kurnool and Oriental tobaccos, including Katerini, Prelip, Komotini, Xanthi and Yambol tobaccos), Maryland, dark, dark-fired, dark air cured (e.g., Madole, Passanda, Cubano, Jatin and Bezuki tobaccos), light air cured (e.g., North Wisconsin and Galpao tobaccos), Indian air cured, Red Russian and Rustica tobaccos, as well as various other rare or specialty tobaccos and various blends of any of the foregoing tobaccos. Tobacco material may also have a so-called “blended” form. For example, the tobacco material may include a mixture of parts or pieces of flue-cured, burley (e.g., Malawi burley tobacco) and Oriental tobaccos (e.g., as tobacco composed of, or derived from, tobacco lamina, or a mixture of tobacco lamina and tobacco stem).

Tobacco materials used as components in the present disclosure can be subjected to, for example, fermentation, bleaching, and the like. If desired, the tobacco materials can be, for example, irradiated, pasteurized, or otherwise subjected to controlled heat treatment. Such treatment processes are detailed, for example, in U.S. Pat. No. 8,061,362 to Mua et al., which is incorporated herein by reference. In certain embodiments, tobacco materials can be treated with water and an additive capable of inhibiting reaction of asparagine to form acrylamide upon heating of the tobacco material (e.g., an additive selected from the group consisting of lysine, glycine, histidine, alanine, methionine, cysteine, glutamic acid, aspartic acid, proline, phenylalanine, valine, arginine, compositions incorporating di- and trivalent cations, asparaginase, certain non-reducing saccharides, certain reducing agents, phenolic compounds, certain compounds having at least one free thiol group or functionality, oxidizing agents, oxidation catalysts, natural plant extracts (e.g., rosemary extract), and combinations thereof. See, for example, the types of treatment processes described in U.S. Pat. Nos. 8,434,496, 8,944,072, and 8,991,403 to Chen et al., which are all incorporated herein by reference.

Various representative tobacco types, processed types of tobaccos, and types of tobacco blends are set forth in U.S. Pat. No. 4,836,224 to Lawson et al.; U.S. Pat. No. 4,924,888 to Perfetti et al.; U.S. Pat. No. 5,056,537 to Brown et al.; U.S. Pat. No. 5,159,942 to Brinkley et al.; U.S. Pat. No. 5,220,930 to Gentry; U.S. Pat. No. 5,360,023 to Blakley et al.; U.S. Pat. No. 6,701,936 to Shafer et al.; U.S. Pat. No. 7,011,096 to Li et al.; and U.S. Pat. No. 7,017,585 to Li et al.; U.S. Pat. No. 7,025,066 to Lawson et al.; U.S. Pat. App. Pub. No. 2004-0255965 to Perfetti et al.; PCT Pat. App. Pub. No. WO 02/37990 to Bereman; and Bombick et al., Fund. Appl. Toxicol., 39, p. 11-17 (1997); which are incorporated herein by reference in their entireties.

The quantity of tobacco material, if present within the alginate matrix of the present disclosure, may vary. When the component-containing, alginate-based substrates provided herein comprise one or more tobacco materials, typical inclusion ranges for tobacco materials can vary depending on the nature and type of the tobacco material, and the intended effect on the final mixture/substrate, with an example range of up to about 80% tobacco material by weight, up to about 70%, up to about 60%, up to about 50%, up to about 40%, or up to about 30% by weight (or up to about 20% by weight or up to about 10% by weight or up to about 5% by weight), based on total weight of the mixture (e.g., about 0.1 to about 15% by weight). Certain exemplary ranges include about 50% to about 80% by dry weight.

While some mixtures may contain tobacco or tobacco-derived materials (e.g., tobacco extracts and/or particulate tobacco), in other embodiments, the mixture of step 10 (and, correspondingly, the component-containing alginate-based substrate) can be characterized as completely free or substantially free of tobacco material (other than purified nicotine as an active ingredient). By “substantially free” of tobacco-derived materials is meant that no tobacco-derived material has been intentionally added, beyond trace amounts that may be naturally present in e.g., another botanical or plant-derived material. For example, certain embodiments can be characterized as having less than 1% by weight, or less than 0.5% by weight, or less than 0.1% by weight of tobacco material, or 0% by weight of tobacco material.

A further example of a component of the mixture of step 10 provided herein is an active agent (also referred to herein as an “active ingredient”). The active ingredient can be any known agent adapted for therapeutic, prophylactic, or diagnostic use. These can include, for example, synthetic organic compounds, proteins and peptides, polysaccharides and other sugars, lipids, inorganic compounds, and nucleic acid sequences, having therapeutic, prophylactic, or diagnostic activity.

As used herein, an “active ingredient” refers to one or more substances belonging to any of the following categories: API (active pharmaceutical ingredient), food additives, natural medicaments, and naturally occurring substances that can have an effect on humans. Example active ingredients include any ingredient known to impact one or more biological functions within the body, such as ingredients that furnish pharmacological activity or other direct effect in the diagnosis, cure, mitigation, treatment, or prevention of disease, or which affect the structure or any function of the body of humans (e.g., provide a stimulating action on the central nervous system, have an energizing effect, an antipyretic or analgesic action, or an otherwise useful effect on the body). In some embodiments, the active ingredient may be of the type generally referred to as dietary supplements, nutraceuticals, “phytochemicals” or “functional foods.” These types of additives are sometimes defined in the art as encompassing substances typically available from naturally-occurring sources (e.g., botanical materials) that provide one or more advantageous biological effects (e.g., health promotion, disease prevention, or other medicinal properties), but are not classified or regulated as drugs.

Non-limiting examples of active ingredients include those falling in the categories of botanical/herbal ingredients (e.g., hemp, eucalyptus, rooibos, fennel, lemongrass, fennel, citrus, cloves, lavender, peppermint, flax, chamomile, basil, rosemary, ginger, turmeric, Ginkgo biloba, hazel, laurel, green tea, white mulberry, cannabis, cocoa, ashwagandha, baobab, chlorophyll, cordyceps, damiana, ginseng, guarana, maca, and tisanes), stimulants (e.g., caffeine and guarana), amino acids (e.g., taurine, theanine, phenylalanine, tyrosine, and tryptophan), nicotine components, and/or pharmaceutical, nutraceutical, and medicinal ingredients (e.g., melatonin, vitamins such as A, B3, B6, B12, and C, and/or cannabinoids, such as tetrahydrocannabinol (THC) and cannabidiol (CBD)). Each of these categories is further described herein below.

In certain embodiments, the active ingredient is selected from the group consisting of caffeine, taurine, GABA, theanine, vitamin C, lemon balm extract, ginseng, citicoline, sunflower lecithin, and combinations thereof. For example, the active ingredient can include a combination of caffeine, theanine, and optionally ginseng. In another embodiment, the active ingredient includes a combination of theanine, gamma-amino butyric acid (GABA), and lemon balm extract. In a further embodiment, the active ingredient includes theanine, theanine and tryptophan, or theanine and one or more B vitamins (e.g., vitamin B6 or B12). In a still further embodiment, the active ingredient includes a combination of caffeine, taurine, and vitamin C.

The particular percentages and choice of ingredients will vary depending upon the desired flavor, texture, and other characteristics. Example active ingredients would include any ingredient known to impact one or more biological functions within the body, such as ingredients that furnish pharmacological activity or other direct effect in the diagnosis, cure, mitigation, treatment, or prevention of disease, or which affect the structure or any function of the body of humans or other animals (e.g., provide a stimulating action on the central nervous system, have an energizing effect, an antipyretic or analgesic action, or an otherwise useful effect on the body). Typically, an active ingredient or combination thereof is present in a total concentration of at least about 0.001% by weight of the component-containing alginate-based substrate, such as in a range from about 0.001% to about 20%. In some embodiments, the active ingredient or combination of active ingredients is present in a concentration from about 0.1% w/w to about 10% by weight, such as, e.g., from about 0.5% w/w to about 10%, from about 1% to about 10%, from about 1% to about 5% by weight, based on the total weight of the component-containing alginate-based substrate. In some embodiments, the active ingredient or combination of active ingredients is present in a concentration of from about 0.001%, about 0.01%, about 0.1%, or about 1%, up to about 20% by weight, such as, e.g., from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the component-containing alginate-based substrate. When the component-containing, alginate-based substrates provided herein comprise one or more active ingredients, the amount can vary based on the type of active ingredient. Further suitable ranges for specific active ingredients are provided herein below.

### Botanical

In some embodiments, the active ingredient may comprise a botanical ingredient. As used herein, the term “botanical ingredient” or “botanical” refers to any plant material or fungal-derived material, including plant material in its natural form and plant material derived from natural plant materials, such as extracts or isolates from plant materials or treated plant materials (e.g., plant materials subjected to heat treatment, fermentation, bleaching, or other treatment processes capable of altering the physical and/or chemical nature of the material). The term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. For the purposes of the present disclosure, a “botanical material” includes, but is not limited to, an “herbal material,” which refers to seed-producing plants that do not develop persistent woody tissue and are often valued for their medicinal or sensory characteristics (e.g., teas or tisanes). In some embodiments, reference to a botanical material may include tobacco, for example, as described herein above with regard to tobacco-derived botanical particle fines. However, in some embodiments, the botanical material may not be derived from tobacco and may expressly exclude tobacco materials (i.e., does not include any Nicotiana species); such botanical materials can be referred to as “non-tobacco.” For example, in some embodiments, the compositions as disclosed herein can be characterized as free of any tobacco material (e.g., any embodiment as disclosed herein may be completely or substantially free of any tobacco material). By “substantially free” is meant that no tobacco material has been intentionally added. For example, certain embodiments can be characterized as having less than 0.001% by weight of tobacco, or less than 0.0001%, or even 0% by weight of tobacco.

When present, a botanical is typically at a concentration of from about 0.01% w/w to about 10% by weight, such as, e.g., from about 0.01% w/w, about 0.05%, about 0.1%, or about 0.5%, to about 1%, about 2%, about 3%, about 4%, about 5%, about 6%, about 7%, about 8%, about 9%, or about 10%, about 11%, about 12%, about 13%, about 14%, or about 15% by weight, based on the total weight of the component-containing alginate-based substrate.

The botanical materials useful in the present disclosure may comprise, without limitation, any of the compounds and sources set forth herein, including mixtures thereof. Certain botanical materials of this type are sometimes referred to as dietary supplements, nutraceuticals, “phytochemicals” or “functional foods.” Certain botanicals, as the plant material or an extract thereof, have found use in traditional herbal medicine, and are described further herein. Non-limiting examples of botanicals or botanical-derived materials include acai berry, alfalfa, allspice, annatto seed, apricot oil, ashwagandha, Bacopa monniera, baobab, basil, bay leaves, bee balm, beefsteak plant, beet root, wild bergamot, black pepper, black tea, black cohosh, blueberries, borage seed oil, bugleweed, cacao, calamus root, cannabis, carvi, cassis, catnip, catauba, cayenne pepper, Centella asiatica, chaga mushroom, Chai-hu, chamomile, cherry blossom, chervil, chive, chlorophyll, cilantro, cinnamon, citrus, cloves, cocoa, coffee, comfrey leaf and root, cordyceps, coriander, cranberry, curcumin, curcuma, cumin, damiana, dandelion, dark chocolate, Dorstenia arifolia, Dorstenia odorata, Echinacea, elderflower, essential oils, eucalyptus, evening primrose, fennel, feverfew, Galphimia glauca, garlic, geranium, ginger, Ginkgo biloba, ginseng (e.g., Panax ginseng), goji berries, goldenseal, grapefruit, grape seed, green tea, Griffonia simplicifolia, guarana, gutu kola, hawthorn, hemp, hibiscus flower, honeybush, hops, jasmine, jiaogulan, juniper, Kaempferia parviflora (Thai ginseng), kava, lavender, lemon balm, lemon basil, lemon peel, lemongrass, licorice, lutein, maca, mace, marjoram, matcha, milk thistle, mints (e.g., Mentha arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens), myrtle, Nardostachys chinensis, nutmeg, oil-based extract of Viola odorata, oolong tea, olive, orange, orange skin, orange blossom, oregano, papaya, paprika, pennyroyal, peppermint, pimento, potato peel, quercetin, red clover, resveratrol, Rhizoma gastrodiae, Rhodiola, rooibos (red or green), rose essential oil, rosehip, rosemary, sage, clary sage, Saint John's wort, saffron, savory, saw palmetto, Sceletium tortuosum, Schisandra, Skullcap, spearmint extract, Spikenard, spirulina, slippery elm bark, sorghum bran hi-tannin, sorghum grain hi-tannin, sumac bran, tarragon, terpenes, theacrine, thyme, tisanes, turmeric, Turnera aphrodisiaca, uva ursi, valerian, vanilla, verbena, white mulberry, wild yam root, wintergreen, yacon root, yellow dock, Yerba mate, Yerba santa, Bacopa monniera, Withania somnifera, Lion's mane, and Silybum marianum.

In some embodiments, the active ingredient comprises lemon balm. Lemon balm (Melissa officinalis) is a mildly lemon-scented herb from the same family as mint (Lamiaceae). The herb is native to Europe, North Africa, and West Asia. The tea of lemon balm, as well as the essential oil and the extract, are used in traditional and alternative medicine. In some embodiments, the active ingredient comprises lemon balm extract. In some embodiments, the lemon balm extract is present in an amount of from about 1 to about 4% by weight, based on the total weight of the material.

In some embodiments, the active ingredient comprises ginseng. Ginseng is the root of plants of the genus Panax, which are characterized by the presence of unique steroid saponin phytochemicals (ginsenosides) and gintonin. Ginseng finds use as a dietary supplement in energy drinks or herbal teas, and in traditional medicine. Cultivated species include Korean ginseng (P. ginseng), South China ginseng (P. notoginseng), and American ginseng (P. quinquefolius). American ginseng and Korean ginseng vary in the type and quantity of various ginsenosides present. In some embodiments, the ginseng is American ginseng or Korean ginseng. In specific embodiments, the active ingredient comprises Korean ginseng. In some embodiments, ginseng is present in an amount of from about 0.4 to about 0.6% by weight, based on the total weight of the material.

In some embodiments, the active ingredient comprises or is derived from one or more botanicals or constituents, derivatives or extracts thereof, and the botanical is selected from eucalyptus, star anise, cocoa and hemp.

In some embodiments, the active ingredient comprises or is derived from one or more botanicals or constituents, derivatives or extracts thereof, and the botanical is selected from rooibos and fennel. In some embodiments, a combination of tobacco and one or more other plant materials, e.g., the types of plant materials disclosed above (such as botanical materials), may be included as the “component.” In such embodiments, the total amount of tobacco material and other plant material can be up to about 80% by dry weight of the substrate (and, correspondingly, in the slurry/mixture provided by Step 10).

### Stimulants

In some embodiments, the active ingredient comprises one or more stimulants. As used herein, the term “stimulant” refers to a material that increases activity of the central nervous system and/or the body, for example, enhancing focus, cognition, vigor, mood, alertness, and the like. Non-limiting examples of stimulants include caffeine, theacrine, theobromine, and theophylline. Theacrine (1,3,7,9-tetramethyluric acid) is a purine alkaloid which is structurally related to caffeine, and possesses stimulant, analgesic, and anti-inflammatory effects. Present stimulants may be natural, naturally derived, or wholly synthetic. For example, certain botanical materials (guarana, tea, coffee, cocoa, and the like) may possess a stimulant effect by virtue of the presence of e.g., caffeine or related alkaloids, and accordingly are “natural” stimulants. By “naturally derived” is meant the stimulant (e.g., caffeine, theacrine) is in a purified form, outside its natural (e.g., botanical) matrix. For example, caffeine can be obtained by extraction and purification from botanical sources (e.g., tea). By “wholly synthetic”, it is meant that the stimulant has been obtained by chemical synthesis. In some embodiments, the active ingredient comprises caffeine. In some embodiments, the caffeine is present in an encapsulated form. On example of an encapsulated caffeine is Vitashure®, available from Balchem Corp., 52 Sunrise Park Road, New Hampton, N.Y., 10958.

When present, a stimulant or combination of stimulants (e.g., caffeine, theacrine, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the material. In some embodiments, the composition comprises caffeine in an amount of from about 1.5 to about 6% by weight, based on the total weight of the component-containing alginate-based substrate.

### Amino Acids

In some embodiments, the active ingredient comprises an amino acid. As used herein, the term “amino acid” refers to an organic compound that contains amine (—NH₂) and carboxyl (—COOH) or sulfonic acid (SO₃H) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein, or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, and beta-alanine. In some embodiments, the active ingredient comprises theanine. In some embodiments, the active ingredient comprises GABA. In some embodiments, the active ingredient comprises a combination of theanine and GABA. In some embodiments, the active ingredient is a combination of theanine, GABA, and lemon balm. In some embodiments, the active ingredient is a combination of caffeine, theanine, and ginseng. In some embodiments, the active ingredient comprises taurine. In some embodiments, the active ingredient is a combination of caffeine and taurine.

When present, an amino acid or combination of amino acids (e.g., theanine, GABA, and combinations thereof) is typically at a concentration of from about 0.1% w/w to about 15% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, or about 15% by weight, based on the total weight of the component-containing alginate-based substrate.

### Vitamins

In some embodiments, the active ingredient comprises a vitamin or combination of vitamins. As used herein, the term “vitamin” refers to an organic molecule (or related set of molecules) that is an essential micronutrient needed for the proper functioning of metabolism in a mammal. There are thirteen vitamins required by human metabolism, which are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). In some embodiments, the active ingredient comprises vitamin C. In some embodiments, the active ingredient is a combination of vitamin C, caffeine, and taurine.

When present, a vitamin or combination of vitamins (e.g., vitamin B6, vitamin B12, vitamin E, vitamin C, or a combination thereof) is typically at a concentration of from about 0.01% w/w to about 6% by weight, such as, e.g., from about 0.01%, about 0.02%, about 0.03%, about 0.04%, about 0.05%, about 0.06%, about 0.07%, about 0.08%, about 0.09%, or about 0.1% w/w, to about 0.2%, about 0.3%, about 0.4%, about 0.5% about 0.6%, about 0.7%, about 0.8%, about 0.9%, about 1%, about 2%, about 3%, about 4%, about 5%, or about 6% by weight, based on the total weight of the component-containing alginate-based substrate.

### Antioxidants

In some embodiments, the active ingredient comprises one or more antioxidants. As used herein, the term “antioxidant” refers to a substance which prevents or suppresses oxidation by terminating free radical reactions, and may delay or prevent some types of cellular damage. Antioxidants may be naturally occurring or synthetic. Naturally occurring antioxidants include those found in foods and botanical materials. Non-limiting examples of antioxidants include certain botanical materials, vitamins, polyphenols, and phenol derivatives.

Examples of botanical materials which are associated with antioxidant characteristics include without limitation acai berry, alfalfa, allspice, annatto seed, apricot oil, basil, bee balm, wild bergamot, black pepper, blueberries, borage seed oil, bugleweed, cacao, calamus root, catnip, catuaba, cayenne pepper, chaga mushroom, chervil, cinnamon, dark chocolate, potato peel, grape seed, ginseng, Gingko biloba, Saint John's Wort, saw palmetto, green tea, black tea, black cohosh, cayenne, chamomile, cloves, cocoa powder, cranberry, dandelion, grapefruit, honeybush, echinacea, garlic, evening primrose, feverfew, ginger, goldenseal, hawthorn, hibiscus flower, jiaogulan, kava, lavender, licorice, marjoram, milk thistle, mints (menthe), oolong tea, beet root, orange, oregano, papaya, pennyroyal, peppermint, red clover, rooibos (red or green), rosehip, rosemary, sage, clary sage, savory, spearmint, spirulina, slippery elm bark, sorghum bran hi-tannin, sorghum grain hi-tannin, sumac bran, comfrey leaf and root, goji berries, gutu kola, thyme, turmeric, uva ursi, valerian, wild yam root, wintergreen, yacon root, yellow dock, Yerba mate, Yerba santa, Bacopa monniera, Withania somnifera, Lion's mane, and Silybum marianum. Such botanical materials may be provided in fresh or dry form, essential oils, or may be in the form of an extracts. The botanical materials (as well as their extracts) often include compounds from various classes known to provide antioxidant effects, such as minerals, vitamins, isoflavones, phytoesterols, allyl sulfides, dithiolthiones, isothiocyanates, indoles, lignans, flavonoids, polyphenols, and carotenoids. Examples of compounds found in botanical extracts or oils include ascorbic acid, peanut endocarb, resveratrol, sulforaphane, beta-carotene, lycopene, lutein, co-enzyme Q, carnitine, quercetin, kaempferol, and the like. See, e.g., Santhosh et al., Phytomedicine, 12 (2005) 216-220, which is incorporated herein by reference.

Non-limiting examples of other suitable antioxidants include citric acid, Vitamin E or a derivative thereof, a tocopherol, epicatechol, epigallocatechol, epigallocatechol gallate, erythorbic acid, sodium erythorbate, 4-hexylresorcinol, theaflavin, theaflavin monogallate A or B, theaflavin digallate, phenolic acids, glycosides, quercitrin, isoquercitrin, hyperoside, polyphenols, catechols, resveratrols, oleuropein, butylated hydroxyanisole (BHA), butylated hydroxytoluene (BHT), tertiary butylhydroquinone (TBHQ), and combinations thereof.

When present, an antioxidant is typically at a concentration of from about 0.001% w/w to about 10% by weight, such as, e.g., from about 0.001%, about 0.005%, about 0.01% w/w, about 0.05%, about 0.1%, or about 0.5%, to about 1%, about 2%, about 3%, about 4%, about 5%, about 6%, about 7%, about 8%, about 9%, or about 10%, based on the total weight of the component-containing alginate-based substrate.

### Nicotine Component

In certain embodiments, the active ingredient comprises a nicotine component. By “nicotine component” is meant any suitable form of nicotine (e.g., free base or salt) for providing oral absorption of at least a portion of the nicotine present. Typically, the nicotine component is selected from the group consisting of nicotine free base and a nicotine salt. In some embodiments, the nicotine component is nicotine in its free base form, which easily can be adsorbed in for example, a microcrystalline cellulose material to form a microcrystalline cellulose-nicotine carrier complex. See, for example, the discussion of nicotine in free base form in US Pat. Pub. No. 2004/0191322 to Hansson, which is incorporated herein by reference.

In some embodiments, at least a portion of the nicotine component can be employed in the form of a salt. Salts of nicotine can be provided using the types of ingredients and techniques set forth in U.S. Pat. No. 2,033,909 to Cox et al. and Perfetti, Beitrage Tabakforschung Int., 12: 43-54 (1983), which are incorporated herein by reference. Further examples of nicotine salts are provided in U.S. Pat. Nos. 9,738,622, 9,896,429, and 10,508,096, which are incorporated herein by reference in their entireties. Additionally, salts of nicotine are available from sources such as Pfaltz and Bauer, Inc. and K&K Laboratories, Division of ICN Biochemicals, Inc. Typically, the nicotine component is selected from the group consisting of nicotine free base, a nicotine salt such as hydrochloride, dihydrochloride, monotartrate, bitartrate, sulfate, salicylate, and nicotine zinc chloride. In some embodiments, the nicotine component or a portion thereof is a nicotine salt with one or more organic acids.

In some embodiments, at least a portion of the nicotine can be in the form of a resin complex of nicotine, where nicotine is bound in an ion-exchange resin, such as nicotine polacrilex, which is nicotine bound to, for example, a polymethacrilic acid, such as Amberlite IRP64, Purolite C115HMR, or Doshion P551. See, for example, U.S. Pat. No. 3,901,248 to Lichtneckert et al., which is incorporated herein by reference. Another example is a nicotine-polyacrylic carbomer complex, such as with Carbopol 974P. In some embodiments, nicotine may be present in the form of a nicotine polyacrylic complex.

Typically, the nicotine component (calculated as the free base) when present, is in a concentration of at least about 0.001% by weight of the component-containing alginate-based substrate, such as in a range from about 0.001% to about 10%. In some embodiments, the nicotine component is present in a concentration from about 0.1% w/w to about 10% by weight, such as, e.g., from about 0.1% w/w, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, or about 10% by weight, calculated as the free base and based on the total weight of the component-containing alginate-based substrate. In some embodiments, the nicotine component is present in a concentration from about 0.1% w/w to about 7.5% by weight, such as, e.g., from about from about 0.1% w/w to about 2.5%, from about 0.1% to about 2.0%, from about 0.1% to about 1.5%, or from about 0.1% to about 6% by weight, calculated as the free base and based on the total dry weight of the mixture. In some embodiments, the nicotine component is present in a concentration from about 0.1% w/w to about 3% by weight, such as, e.g., from about 0.1% w/w to about 2.5%, from about 0.1% to about 2.0%, from about 0.1% to about 1.5%, or from about 0.1% to about 1% by weight, calculated as the free base and based on the total weight of the component-containing alginate-based substrate. It is also noted that nicotine may be introduced via tobacco material, as referenced above, and in some embodiments, the inclusion of tobacco as a component in the methods and materials provided herein may provide a total nicotine content in the slurry/substrate within the ranges disclosed herein.

In some embodiments, the products or compositions of the disclosure can be characterized as free of any nicotine component (e.g., any embodiment as disclosed herein may be completely or substantially free of any nicotine component). By “substantially free” is meant that no nicotine has been intentionally added, beyond trace amounts that may be naturally present in e.g., a botanical material. For example, certain embodiments can be characterized as having less than 0.001% by weight of nicotine, or less than 0.0001%, or even 0% by weight of nicotine, calculated as the free base. In some embodiments, the active ingredient comprises a nicotine component (e.g., any product or composition of the disclosure, in addition to comprising any active ingredient or combination of active ingredients as disclosed herein, may further comprise a nicotine component).

### Cannabinoids

In some embodiments, the active ingredient comprises one or more cannabinoids. As used herein, the term “cannabinoid” refers to a class of diverse chemical compounds that acts on cannabinoid receptors, also known as the endocannabinoid system, in cells that alter neurotransmitter release in the brain. Ligands for these receptor proteins include the endocannabinoids produced naturally in the body by animals; phytocannabinoids, found in cannabis; and synthetic cannabinoids, manufactured artificially. Cannabinoids found in cannabis include, without limitation: cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), and tetrahydrocannabivarinic acid (THCV A). In certain embodiments, the cannabinoid is selected from tetrahydrocannabinol (THC), the primary psychoactive compound in cannabis, and cannabidiol (CBD) another major constituent of the plant, but which is devoid of psychoactivity. All of the above compounds can be used in the form of an isolate from plant material or synthetically derived.

Alternatively, the active ingredient can be a cannabimimetic, which is a class of compounds derived from plants other than cannabis that have biological effects on the endocannabinoid system similar to cannabinoids. Examples include yangonin, alpha-amyrin or beta-amyrin (also classified as terpenes), cyanidin, curcumin (turmeric), catechin, quercetin, salvinorin A, N-acylethanolamines, and N-alkylamide lipids.

When present, a cannabinoid (e.g., CBD) or cannabimimetic is typically in a concentration of at least about 0.1% by weight of the component-containing alginate-based substrate, such as in a range from about 0.1% to about 30%, such as, e.g., from about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 15%, about 20%, or about 30% by weight, based on the total weight of the component-containing alginate-based substrate.

### Terpenes

Active ingredients suitable for use in the present disclosure can also be classified as terpenes, many of which are associated with biological effects, such as calming effects. Terpenes are understood to have the general formula of (C₅H₈)ₙ and include monoterpenes, sesquiterpenes, and diterpenes. Terpenes can be acyclic, monocyclic or bicyclic in structure. Some terpenes provide an entourage effect when used in combination with cannabinoids or cannabimimetics. Examples include beta-caryophyllene, linalool, limonene, beta-citronellol, linalyl acetate, pinene (alpha or beta), geraniol, carvone, eucalyptol, menthone, iso-menthone, piperitone, myrcene, beta-bourbonene, and germacrene, which may be used singly or in combination.

### Pharmaceutical Ingredients

In some embodiments, the active ingredient comprises an active pharmaceutical ingredient (API). The API can be any known agent adapted for therapeutic, prophylactic, or diagnostic use. These can include, for example, synthetic organic compounds, proteins and peptides, polysaccharides and other sugars, lipids, phospholipids, inorganic compounds (e.g., magnesium, selenium, zinc, nitrate), neurotransmitters or precursors thereof (e.g., serotonin, 5-hydroxytryptophan, oxitriptan, acetylcholine, dopamine, melatonin), and nucleic acid sequences, having therapeutic, prophylactic, or diagnostic activity. Non-limiting examples of APIs include analgesics and antipyretics (e.g., acetylsalicylic acid, acetaminophen, 3-(4-isobutylphenyl)propanoic acid), phosphatidylserine, myoinositol, docosahexaenoic acid (DHA, Omega-3), arachidonic acid (AA, Omega-6), S-adenosylmethionine (SAM), beta-hydroxy-beta-methylbutyrate (HMB), citicoline (cytidine-5′-diphosphate-choline), and cotinine. In some embodiments, the active ingredient comprises citicoline. In some embodiments, the active ingredient is a combination of citicoline, caffeine, theanine, and ginseng. In some embodiments, the active ingredient comprises sunflower lecithin. In some embodiments, the active ingredient is a combination of sunflower lecithin, caffeine, theanine, and ginseng.

The amount of API may vary. For example, when present, an API is typically at a concentration of from about 0.001% w/w to about 10% by weight, such as, e.g., from about 0.01%, about 0.02%, about 0.03%, about 0.04%, about 0.05%, about 0.06%, about 0.07%, about 0.08%, about 0.09%, about 0.1% w/w, about 0.2%, about 0.3%, about 0.4%, about 0.5% about 0.6%, about 0.7%, about 0.8%, about 0.9%, or about 1%, to about 2%, about 3%, about 4%, about 5%, about 6%, about 7%, about 8%, about 9%, or about 10% by weight, based on the total weight of the component-containing alginate-based substrate.

In some embodiments, the component-containing alginate-based substrate is substantially free of any API. By “substantially free of any API” means that the composition does not contain, and specifically excludes, the presence of any API as defined herein, such as any Food and Drug Administration (FDA) approved therapeutic agent intended to treat any medical condition.

A further example of a component that can be incorporated within an alginate matrix according to the disclosed method is a sweetener. Sweeteners can be used in natural or artificial form or as a combination of artificial and natural sweeteners. Examples of natural sweeteners include fructose, sucrose, glucose, maltose, dextrose, fructose, mannose, galactose, lactose, stevia, honey, and the like. Examples of artificial sweeteners include sucralose, isomaltulose, maltodextrin, saccharin, aspartame, acesulfame K, neotame and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates).

When present, a sweetener or combination of sweeteners may make up from about 0.1 to about 20% or more of the of the mixture 10 by dry weight, for example, from about 0.1 to about 1%, from about 1 to about 5%, from about 5 to about 10%, or from about 10 to about 20% by weight, based on the total dry weight of the mixture. In some embodiments, a combination of sweeteners is present at a concentration of from about 1% to about 3% by dry weight of the mixture.

Another example of a component that can be incorporated within an alginate matrix is an aerosol forming agent. Aerosol forming agents (also referred to as “aerosol formers” or “humectants”) are components with the ability to yield visible aerosols when vaporized upon exposure to heat under those conditions experienced during normal use of atomizers that are characteristic of the current disclosure. The aerosol forming material may include one or more of water, polyhydric alcohols, polysorbates, sorbitan esters, fatty acids, fatty acid esters, waxes, terpenes, sugar alcohols, active ingredients, or a combination thereof. Aerosol forming agents include humectants, e.g., glycerin, propylene glycol, and the like. Other example aerosol forming agents include diethylene glycol, triethylene glycol, tetraethylene glycol, 1,3-butylene glycol, erythritol, meso-erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

In some embodiments, the aerosol forming materials comprise one or more polysorbates. Examples of polysorbates include Polysorbate 60 (polyoxyethylene (20) sorbitan monostearate, Tween 60) and Polysorbate 80 (polyoxyethylene (20) sorbitan monooleate, Tween 80). The type of polysorbate used or the combination of polysorbates used depends on the intended effect desired, as the different polysorbates offer different attributes due to molecular sizes. For example, the polysorbate molecules increase in size from Polysorbate 20 to Polysorbate 80. Using smaller size polysorbate molecules creates less vapor quantity, but permits deeper lung penetration. This may be desirable when the user is in public where he would not want to create a large plume of “smoke” (i.e. vapors). Conversely, if a dense vapor is desired, which can convey the aromatic constituents of tobacco, larger polysorbate molecules can be employed. An additional benefit of using the polysorbate family of compounds is that the polysorbates lower the heat of vaporization of mixtures in which they are present.

In some embodiments, the aerosol forming materials comprise one or more sorbitan esters. Examples of sorbitan esters include sorbitan monolaurate, sorbitan monostearate (Span 60), sorbitan monooleate (Span 20), and sorbitan tristearate (Span 65). In some embodiments, the aerosol forming materials comprise one or more fatty acids. Fatty acids may include short-chain, long-chain, saturated, unsaturated, straight chain, or branched chain carboxylic acids. Fatty acids generally include C4 to C28 aliphatic carboxylic acids. Non-limiting examples of short- or long-chain fatty acids include butyric, propionic, valeric, oleic, linoleic, stearic, myristic, and palmitic acids. In some embodiments, the aerosol forming materials comprise one or more fatty acid esters. Examples of fatty acid esters include alkyl esters, monoglycerides, diglycerides, and triglycerides. Examples of monoglycerides include monolaurin and glycerol monostearate. Examples of triglycerides include triolein, tripalmitin, tristearate, glycerol tributyrate, and glycerol trihexanoate). In some embodiments, the aerosol forming materials comprise one or more waxes. Examples of waxes include carnauba, beeswax, candellila, which are known known to stabilize aerosol particles, improve palatability, or reduce throat irritation. In some embodiments, the aerosol forming materials comprise one or more cannabinoids. In some embodiments, the cannabinoid comprises cannabidiol (CBD), tetrahydrocannabinol (THC), or a combination thereof. In some embodiments, the aerosol forming materials comprise one or more terpenes. As used herein, the term “terpenes” refers to hydrocarbon compounds produced by plants biosynthetically from isopentenyl pyrophosphate. Non-limiting examples of terpenes include limonene, pinene, farnesene, myrcene, geraniol, fennel, and cembrene. In some embodiments, the aerosol forming materials comprise one or more sugar alcohols. Examples of sugar alcohols include sorbitol, erythritol, mannitol, maltitol, isomalt, and xylitol. Sugar alcohols may also serve as flavor enhancers to certain flavor compounds, e.g. menthol and other volatiles, and generally improve on mouthfeel, tactile sensation, throat impact, and other sensory properties, of the resulting aerosol. Sugar alcohols are as referenced above with respect to sweeteners.

When present, an aerosol former may make up from about 1 to about 60% of the of the mixture 10 by dry weight, for example, from about 10% to about 60%, about 20% to about 60%, about 10% to about 40% or from about 15% to about 30% by dry weight, and in one specific example, about 20% by dry weight, based on the total dry weight of the mixture (and, correspondingly, the final substrate).

A still further example of a component that can be incorporated within an alginate matrix according to the present disclosure is a filler. Fillers may comprise materials such as starches, sugars, sugar alcohols, wood fibers/wood pulp, inorganic substances, inert materials, and the like. In some embodiments, the at least one filler comprises a starch, including native and modified starches. “Starch” as used herein may refer to pure starch from any source, modified starch, or starch derivatives. Starch is present, typically in granular form, in almost all green plants and in various types of plant tissues and organs (e.g., seeds, leaves, rhizomes, roots, tubers, shoots, fruits, grains, and stems). Starch can vary in composition, as well as in granular shape and size. Often, starch from different sources has different chemical and physical characteristics. A specific starch can be selected for inclusion in the beads based on the ability of the starch material to impart a specific organoleptic property to the beads. Starches derived from various sources can be used. For example, major sources of starch include cereal grains (e.g., rice, wheat, and maize) and root vegetables (e.g., potatoes and cassava). Other examples of sources of starch include acorns, arrowroot, arracacha, bananas, barley, beans (e.g., favas, lentils, mung beans, peas, chickpeas), breadfruit, buckwheat, canna, chestnuts, colacasia, katakuri, kudzu, malanga, millet, oats, oca, Polynesian arrowroot, sago, sorghum, sweet potato, quinoa, rye, tapioca, taro, tobacco, water chestnuts, and yams. Suitable starches include, but are not limited to, corn starch, rice starch, and modified food starches. Certain starches are modified starches. A modified starch has undergone one or more structural modifications, often designed to alter its high heat properties. Some starches have been developed by genetic modifications, and are considered to be “modified” starches. Other starches are obtained and subsequently modified. For example, modified starches can be starches that have been subjected to chemical reactions, such as esterification, etherification, oxidation, depolymerization (thinning) by acid catalysis or oxidation in the presence of base, bleaching, transglycosylation and depolymerization (e.g., dextrinization in the presence of a catalyst), cross-linking, enzyme treatment, acetylation, hydroxypropylation, and/or partial hydrolysis. Other starches are modified by heat treatments, such as pregelatinization, dextrinization, and/or cold water swelling processes. Certain modified starches include monostarch phosphate, distarch glycerol, distarch phosphate esterified with sodium trimetaphosphate, phosphate distarch phosphate, acetylated distarch phosphate, starch acetate esterified with acetic anhydride, starch acetate esterified with vinyl acetate, acetylated distarch adipate, acetylated distarch glycerol, hydroxypropyl starch, hydroxypropyl distarch glycerol, and starch sodium octenyl succinate.

Fillers can comprise, e.g., corn starch, rice starch, modified food starch, dextran, cyclodextran, or a combination thereof. In some embodiments, fillers comprise a sugar or sugar alcohol (as referenced above as suitable sweeteners as well). In some embodiments, a filler comprises a cellulose material, such as microcrystalline cellulose (“mcc”). The mcc may be synthetic or semi-synthetic, or it may be obtained entirely from natural celluloses. The mcc may be selected from the group consisting of AVICEL® grades PH-100, PH-102, PH-103, PH-105, PH-112, PH-113, PH-200, PH-300, PH-302, VIVACEL® grades 101, 102, 12, 20 and EMOCEL® grades 50M and 90M, and the like, and mixtures thereof. Fillers can comprise wood fibers. In some embodiments, fillers comprise inorganic substances or inert substances, such as, but not limited to, chitosan, carbons (graphite, diamond, fullerenes, graphene), quartz, granite, diatomaceous earth, calcium carbonate, calcium phosphate, clays, crustacean and other marine shells, or combinations thereof. Certain example fillers include maltodextrin, dextrose, calcium carbonate, calcium phosphate, lactose, sugar alcohols, microcrystalline cellulose, or a combination thereof.

The amount of filler can vary, but is typically greater than about 10%, and up to about 90% of the mixture 10 by dry weight. A typical range of filler within the mixture can be from about 10% to about 90% by dry weight of the mixture, for example, from about 10%, about 20%, about 25%, or about 30%, to about 35%, about 40%, about 45%, about 50%, about 60%, about 70%, about 80%, or about 90% by dry weight (e.g., about 20% to about 50%, or about 50% to about 90% by dry weight). In certain embodiments, the amount of filler is at least about 10% by weight, such as at least about 50%, or at least about 70%, or at least about 80%, based on the total dry weight of mixture 10 (and, correspondingly, the final substrate).

### Crosslinking—Step 12

Step 12 of the method comprises subjecting the mixture provided via step 10 to cross-linking. Alginates, as referenced above, comprise carboxylate groups on the polymer backbone and in the presence of various divalent or trivalent cations, adjacent carboxylate groups on alginate molecules can be crosslinked, typically via ionic interactions (commonly between G-blocks of the polymer chains). As such, alginates with higher G content may result in greater cross-linking, leading to stronger cross-linked products, while alginates with higher M content may result in softer, more fragile cross-linked products.

Example divalent cations suitable for this purpose include, but are not limited to, calcium (Ca²⁺), barium (Ba²⁺), magnesium (Mg²⁺), strontium (Sr²⁺) iron (Fe²⁺), aluminum (Al³⁺), and combinations thereof. The divalent cation(s) can be provided, e.g., in the form of a salt, e.g., including but not limited to a chloride salt, such as CaCl₂. In some embodiments, a suspension of metal carbonate or hydroxycarbonate can be employed, e.g., in a carbon dioxide atmosphere to promote crosslinking (carbon dioxide-induced gelation). Calcium cations are particularly preferred in some embodiments, as in combination with alginates, calcium ions form water-insoluble cross-linked alginates. As such, the formation of water-insoluble cross-linked alginate material can provide an increased solids content in the resulting mixture, e.g., resulting in the formation of a more solid material.

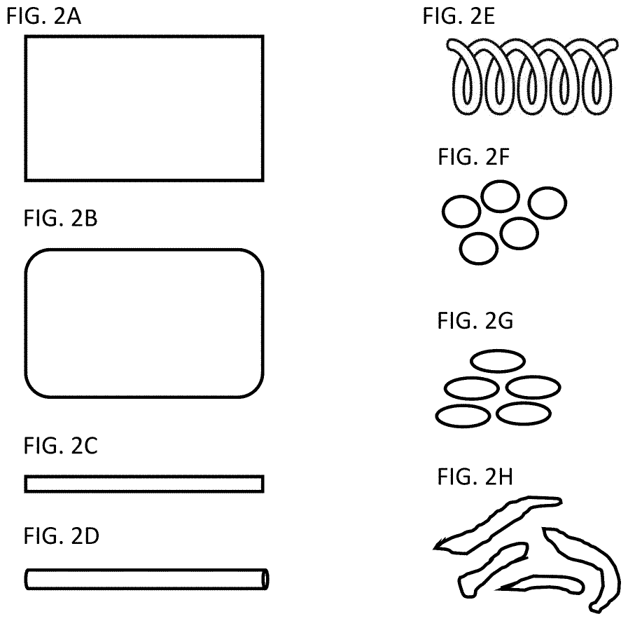

The method by which the mixture of step 10 is crosslinked can vary. The exact method by which the alginate-containing mixture is contacted with the cation(s) can, in some embodiments, determine the form of the final product. Likewise, if a particular form (e.g., shape, size, etc.) of final product is desired, selection of an appropriate method for conducting step 12 should be considered. Shapes of the component-containing, alginate-based substrates described herein can include, e.g., particulate form, shredded form, film form, paper process sheet form, cast sheet form, bead form, granular rod form, or extrudate form, including gels, shreds, films, suspensions, extrusions, shavings, capsules, and/or particles (including pellets, beads, strips, or any desired particle shape of varying sizes) and combinations thereof. Certain examples of shapes that can be provided via the disclosed method are illustrated in FIGS. 2A to 2H and these shapes, as well as methods of providing such shapes, are described in detail herein below.

In some embodiments, the mixture of step 10 is cast into a given shape/form. In some embodiments, the mixture provided by step 10 is cast into a solution comprising the cation into the desired form. Certain such forms include, e.g., the sheets of FIGS. 2A and 2B. The sizes, relative dimensions, and exact shapes of such sheets are not limited; in some embodiments, the dimensions of all sides may be roughly equivalent; in some embodiments, the dimensions of two opposing sides may be longer than those of the other two sides of the sheets (giving a rectangular-type shape); in some embodiments, opposing sides are roughly parallel; in other embodiments, they are not. The thickness of the sheets can also vary, e.g., from a very thin film to a thicker, free-standing sheet. Although the sizes of sheets provided according to the disclosed method can vary, in certain particular embodiments, the sheets are provided so as to be suitable to function as a substrate within a heat-not-burn (HNB) product. In some embodiments, a sheet that is larger than desired is provided and modified to provide the desired size/shape. In some embodiments, the sheet can be a continuous sheet at least about 10 cm wide, such as about 25 cm wide or about 100 cm wide. In some embodiments, sheets can be further processed (e.g., cut, shredded, etc.) into smaller strips, e.g., as shown in FIG. 2C. In some embodiments, such sheets can be gathered or crimped, e.g., to form a cylinder (which can be cut and wrapped to make a consumable material).

In some embodiments, the mixture of step 10 is deposited into a bath (e.g., aqueous solution) of the cation(s). For example, the mixture can be extruded/injected/dropped into a bath comprising the cation, where the rate at which the mixture is extruded/inject/dropped can control the shape/form of the resulting cross-linked material. Such a process can provide, e.g., cylinders (FIG. 2D), noodles (FIG. 2E), spheres (FIG. 2F), elongated spheres (FIG. 2G), and irregular strips (FIG. 2H). It is noted that the “noodle” of FIG. 2E is shown as a perfect spiral; however, more irregular forms of such noodles are also intended to be encompassed herein.

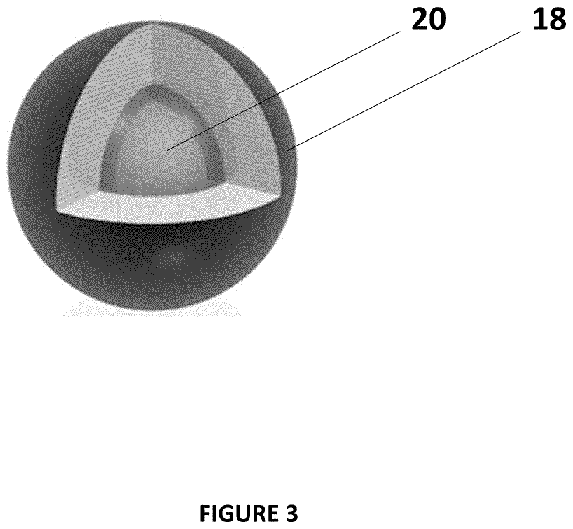

As provided in FIG. 3, in some embodiments, the mixture of step 10 is applied as a coating (18) on a pre-formed material 20 (e.g., a bead, or sphere), wherein the pre-formed material comprises a cation as referenced herein above, suitable for cross-linking the alginate in the coating 18 when it is brought into contact therewith. The mixture can be coated on the pre-formed material 20 in various ways, e.g., via spray-coating, dipping/submerging, adding on top, etc. to provide an “outer shell” comprising the mixture of step 10, which is cross-linked upon/after application to the pre-formed material due to the presence of the cations therein. The pre-formed material 20 can be of varying sizes and shapes (and is not limited, e.g., to a bead/sphere shape as shown and referenced; it may be, for example, a cylindrical form, a square/rectangular form, a sheet, or the like). The alginate-containing coating can coat the entire pre-formed material or a portion of the pre-formed material, and the thickness of the coating can vary. In some embodiments, the pre-formed material contains components (such as those referenced above to be included within the alginate slurry) such as flavorants. In such embodiments, the components (e.g., flavorants) can be effectively encapsulated within the alginate-containing shell. It is noted that in this particular embodiment (where the alginate-containing slurry forms a “shell” around a pre-formed material), the alginate-containing slurry may or may not comprise a component (i.e., the mixture of step 10 may comprise only the alginate and the liquid, e.g., water). In such embodiments, the component(s) can be incorporated solely within the pre-formed material onto which the mixture is coated (or components, which can be the same or different can be incorporated both within the pre-formed material and within the alginate coating).

### Drying—Step 14

Step 14 comprises drying the cross-linked alginate-containing material (e.g., which is typically in the form of a gel). Various drying techniques are known and can be employed in the disclosed method, including but not limited to, evaporative drying, freeze drying, and supercritical drying. Such methods of drying are known; evaporative drying provides mass transfer from the liquid phase (solvent in gel) to the gaseous phase, freeze drying comprises freezing and subliming the solvent, leaving behind a solid material; and supercritical drying generally provides an aerogel. In certain embodiments, the cross-linked alginate material is dried via air drying and/or by heating, e.g., at ambient pressure. Certain, non-limiting methods for drying include fluid bed drying or oven drying. Drying step 14 can, in some embodiments, comprise centrifugation, filtration, or the like.

Drying step 14 can comprise removal of all or a portion of the water associated with the cross-linked alginate material (and can also thus be referred to as “dehydration”). In certain embodiments, it is desirable to maintain some level of moisture in the cross-linked alginate material, e.g., to ensure the material is not too brittle for subsequent applications. It is believed, in some embodiments, that the moisture level may contribute to the cross-linked alginate material's properties with respect to holding the component(s) within the alginate structure and/or releasing such component(s) from the alginate structure. Although these may be described as “dried” materials, it is noted that they nonetheless may comprise some amount of water, and advantageously comprise some amount of water. Examples of relevant moisture contents for the final substrates provided herein may range from about 1% by weight to about 25% by weight, e.g., about 3% by weight to about 20% by weight. In some embodiments, the moisture content of the substrates is about 10 to about 15% by weight (e.g., about 11%).

### Process B

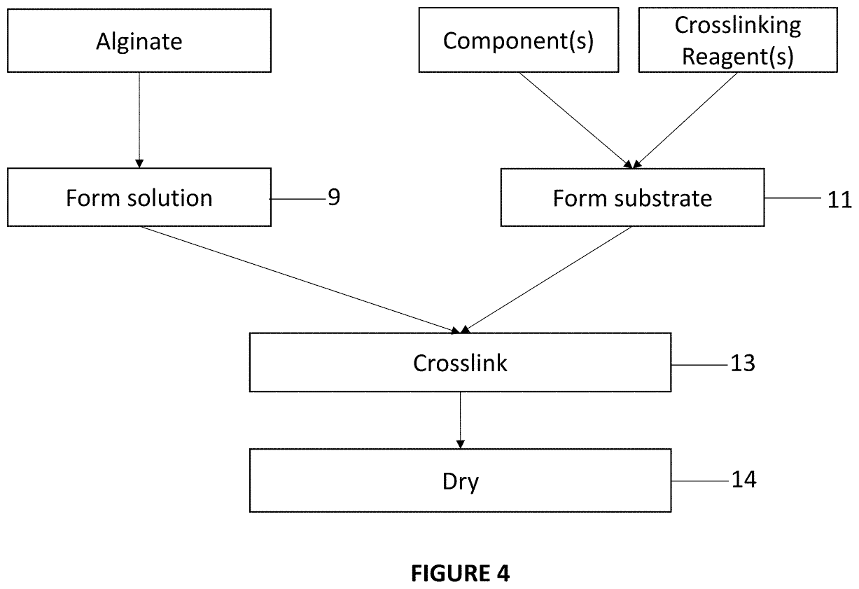

A further method for the temporary entrapment of various components within an alginate polymer matrix according to the present disclosure is shown in FIG. 4 as “Process B.” As shown, step 9 comprises forming a mixture (e.g., solution) of alginate; step 11 comprises separately forming a mixture of a component to be encapsulated within the alginate and divalent or trivalent cation cross-linking reagent(s); step 13 comprises mixing the results of steps 9 and 11, and step 14 comprises drying the resulting material to give a component-containing, alginate-based substrate.

### Solution Formation—Step 9

This step involves the formation of a solution of alginate, by combining an alginate and a liquid (i.e., solvent). Exemplary types of alginates useful in this step are provided above with respect to Process A. The liquid can vary and can be, for example, any liquid referenced herein above, but in certain embodiments, comprises water. The concentration of alginate in the solution formed in Step 9 of Process B can vary widely. In some embodiments, this solution consists essentially only of the alginate and liquid. However, the disclosure is not limited thereto; various other components can optionally be included within the alginate-containing solution (e.g., a further “component,” as referenced above, such as a flavorant and/or an aerosol former).

### Substrate Formation—Step 11

This step involves the formation of a substrate comprising the component to be encapsulated within the alginate (which are described in detail above with respect to mixing step 10 of Process A), as well as the divalent or trivalent cation useful to cross-link the alginate (with exemplary such cations described herein above with respect to step 12 of Process A). In this step 11, the order of addition of components can vary, and the mixture formation is generally facilitated by a liquid. The liquid may be water, but is not limited thereto. The liquid, in some embodiments, comprises one or more ingredients typically included within a substrate as provided herein (e.g., including, but not limited to, fillers, botanical material, tobacco material, aerosol formers, flavors, and any combination thereof).

The exact form of the “mixture” provided via this step can vary widely. In some embodiments, the mixture is a solid or semi-solid material, which comprises sufficient solid material so as to provide a “substrate” in the desired form. The desired form can vary; in some embodiments, the desired form can comprise one of the shapes 2A-2H, depicted in FIG. 2. As such, step 11 in some embodiments comprises not only mixing of the components, but also formation of the components into the desired size/shape of substrate. As such, this step may, in some embodiments, include molding, casting, extruding, etc. to provide a substrate comprising the noted components. The step can, in some embodiments, comprise drying the mixture to some extent, e.g., to provide a substrate that can be suitably manipulated for subsequent steps.

### Cross-Linking—Step 13

The alginate-containing solution provided by Step 9, above, is combined with the substrate (comprising the “component(s)” and the divalent or trivalent cations) in a manner so as to cross-link at least a portion of the alginate within the solution. The method by which the solution is contacted with the substrate in step 13 is not particularly limited. For example, in some embodiments, the substrate is dipped or submerged into the alginate-containing solution; in some embodiments, the alginate-containing solution is sprayed onto or otherwise applied in some manner onto the surface of the substrate. This process of contacting the substrate with the alginate-containing solution advantageously causes the alginate to come into contact with cations in the substrate, thereby cross-linking at least a portion of the alginate present in the solution.

The substrate is advantageously at least partially permeable to the alginate-containing solution, such that the solution can penetrate the substrate and, upon cross-linking, entrapping at least a portion of such components present within the substrate. Where the substrate is less permeable, a “shell” of cross-linked alginate may be formed on the substrate, which can entrap components that were originally present toward the surface of the substrate (as well as components that may optionally be present within the alginate-containing solution).

### Drying—Step 14

Step 14 comprises drying the cross-linked alginate-containing substrate, and parameters and considerations with respect to drying are generally the same as those described above with respect to Process A.

### Applications

The resulting “dried” cross-linked component-containing, alginate-based substrates (from either Process A or Process B) can be used for a range of applications. The substrates can be used in combustible aerosol delivery systems, such as cigarettes, cigarillos, cigars, and tobacco for pipes or for roll-your-own or for make-your-own cigarettes, or non-combustible aerosol delivery systems that release compounds from an aerosol-generating material without combusting the aerosol-generating material, such as electronic cigarettes, tobacco heating products, and hybrid systems to generate aerosol using a combination of aerosol-generating materials. Alternatively, the substrates can be used as a component of aerosol-free delivery systems that deliver an active ingredient or flavor to a user orally, nasally, transdermally or in another way without forming an aerosol, including but not limited to, lozenges, gums, patches, articles comprising inhalable powders, and oral products such as oral tobacco which includes snus or moist snuff, wherein the active ingredient may or may not comprise nicotine. For example, in certain oral products, the components can be released from the alginate matrix, e.g., by chewing or penetration of saliva. Accordingly, it should be understood that the description of the methods and products disclosed herein are discussed in terms of embodiments relating to aerosol delivery devices by way of example only, and may be embodied and used in various other products and methods. According to the present disclosure, a “non-combustible” aerosol delivery system is one where a constituent aerosol-generating material of the aerosol delivery system (or component thereof) is not combusted or burned in order to facilitate delivery of at least one substance to a user. In some embodiments, the delivery system is a non-combustible aerosol delivery system, such as a powered non-combustible aerosol delivery system. In some embodiments, the non-combustible aerosol delivery system is an electronic cigarette, also known as a vaping device or electronic nicotine delivery system (END), although it is noted that the presence of nicotine in the aerosol-generating material is not a requirement. In some embodiments, the non-combustible aerosol delivery system is an aerosol-generating material heating system, also known as a heat-not-burn system. An example of such a system is a tobacco heating system.

In some embodiments, the non-combustible aerosol delivery system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol delivery system may comprise a non-combustible aerosol delivery device and a consumable for use with the non-combustible aerosol delivery device. In some embodiments, the disclosure relates to consumables comprising aerosol-generating material and configured to be used with non-combustible aerosol delivery devices. These consumables are sometimes referred to as articles throughout the disclosure.

In some embodiments, the non-combustible aerosol delivery system, such as a non-combustible aerosol delivery device thereof, may comprise a power source and a controller. The power source may, for example, be an electric power source or an exothermic power source. In some embodiments, the exothermic power source comprises a carbon substrate which may be energized so as to distribute power in the form of heat to an aerosol-generating material or to a heat transfer material in proximity to the exothermic power source. In some embodiments, the non-combustible aerosol delivery system may comprise an area for receiving the consumable, an aerosol generator, an aerosol generation area, a housing, a mouthpiece, a filter and/or an aerosol-modifying agent.

In some embodiments, the consumable for use with the non-combustible aerosol delivery device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

Aerosol delivery devices into which the disclosed alginate-based materials can be incorporated include those generally known in the art. In some embodiments, the disclosed alginate-based materials are incorporated within aerosol-generating devices, e.g., which use electrical energy to heat a material to form an inhalable substance (e.g., electrically heated products) or an ignitable heat source to heat a material (preferably without combusting the material to any significant degree) to form an inhalable substance (e.g., carbon heated products). Components of such systems have the form of articles that are sufficiently compact to be considered hand-held devices. That is, use of components of preferred aerosol delivery devices does not result in the production of smoke in the sense that aerosol results principally from by-products of combustion or pyrolysis of tobacco, but rather, use of those preferred systems results in the production of vapors resulting from volatilization or vaporization of certain components incorporated therein. In some example embodiments, components of aerosol delivery devices may be characterized as electronic cigarettes, and those electronic cigarettes most preferably incorporate tobacco and/or components derived from tobacco, and hence deliver tobacco derived components in aerosol form.

The alginate-based material can advantageously be incorporated within certain such devices, e.g., within an aerosol generating component that includes a substrate portion capable of yielding an aerosol upon application of sufficient heat. The substrate can comprise, at least in part, the component-containing, alginate-based substrate provided herein (such that, in some embodiments, the application as heat affords release of at least one component from the alginate matrix). In some embodiments, the device includes an ignitable heat source configured to heat a substrate material (where the substrate comprises the component-containing, alginate-based substrate and the heat source is capable of generating heat to both release the component(s) from the alginate matrix and aerosolize such components within the substrate.

Aerosol generating components of certain preferred aerosol delivery devices may provide many of the sensations (e.g., inhalation and exhalation rituals, types of tastes or flavors, organoleptic effects, physical feel, use rituals, visual cues such as those provided by visible aerosol, and the like) of smoking a cigarette, cigar or pipe that is employed by lighting and burning tobacco (and hence inhaling tobacco smoke), without any substantial degree of combustion of any component thereof. For example, the user of an aerosol delivery device in accordance with some example embodiments of the present disclosure can hold and use that component much like a smoker employs a traditional type of smoking article, draw on one end of that piece for inhalation of aerosol produced by that piece, take or draw puffs at selected intervals of time, and the like.

While the methods and systems are generally described herein in terms of embodiments associated with aerosol delivery devices and/or aerosol generating components such as so-called “e-cigarettes” or “tobacco heating products,” it should be understood that the mechanisms, components, features, and methods may be embodied in many different forms and associated with a variety of articles. For example, the description provided herein may be employed in conjunction with embodiments of traditional smoking articles (e.g., cigarettes, cigars, pipes, etc.), heat-not-burn cigarettes, and related packaging for any of the products disclosed herein. Accordingly, it should be understood that the description of the mechanisms, components, features, and methods disclosed herein are discussed in terms of embodiments relating to aerosol delivery devices by way of example only, and may be embodied and used in various other products and methods.

Aerosol delivery devices and/or aerosol generating components may also be characterized as being vapor-producing articles or medicament delivery articles. Thus, such articles or devices may be adapted so as to provide one or more substances (e.g., flavors and/or pharmaceutical active ingredients) in an inhalable form or state. For example, inhalable substances may be substantially in the form of a vapor (i.e., a substance that is in the gas phase at a temperature lower than its critical point). Alternatively, inhalable substances may be in the form of an aerosol (i.e., a suspension of fine solid particles or liquid droplets in a gas). For purposes of simplicity, the term “aerosol” as used herein is meant to include vapors, gases and aerosols of a form or type suitable for human inhalation, whether or not visible, and whether or not of a form that might be considered to be smoke-like. The physical form of the inhalable substance may depend upon the nature of the medium and the inhalable substance itself as to whether it exists in a vapor state or an aerosol state. In some embodiments, the terms “vapor” and “aerosol” may be interchangeable. Thus, for simplicity, the terms “vapor” and “aerosol” as used to describe aspects of the disclosure are understood to be interchangeable unless stated otherwise.

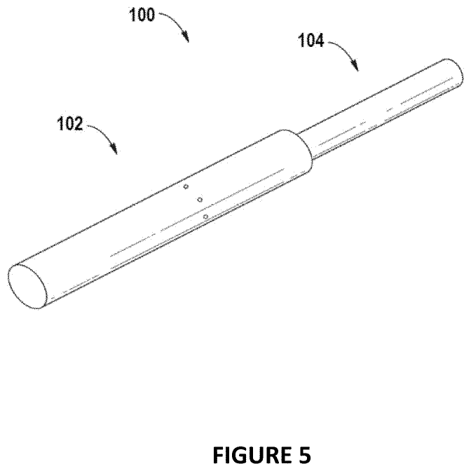

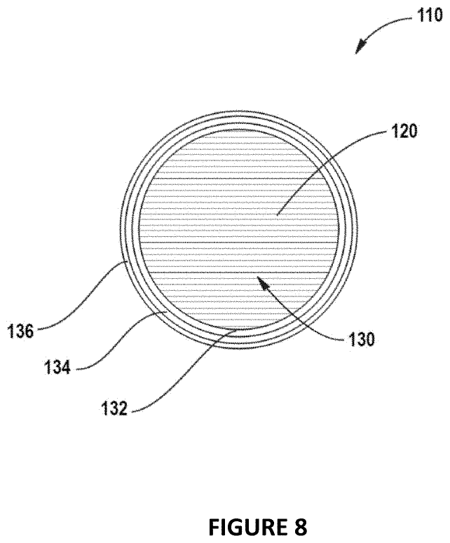

More specific details about aerosol generating components and aerosol delivery devices are disclosed herein below with reference to FIGS. 5 to 10.

### Substrate

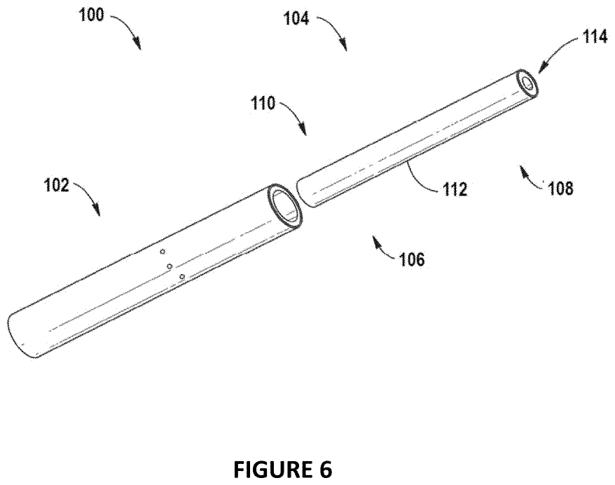

Generally, the aerosol generating components of the present disclosure may be produced via several different methods depending, for example, on the desired composition of the final substrate or the required shape and size of the substrate for a particular aerosol delivery device. Various examples of manufacturing processes and substrate compositions are described herein below. More specific details about aerosol generating components (e.g., substrate 110 in FIGS. 6-8), which can, in some embodiments, comprise an alginate-encapsulated component as provided herein, are disclosed hereinafter with reference to FIGS. 5-8.

In some embodiments, the substrate is in particulate form, shredded form, film form, paper process sheet form, cast sheet form, bead form, granular rod form, or extrudate form. In various embodiments, the form of the substrate may include gels, shreds, films, suspensions, extrusions, shavings, capsules, and/or particles (including pellets, beads, strips, or any desired particle shape of varying sizes) and combinations thereof. In some embodiments, the substrate is formed into a substantially cylindrical shape. In some embodiments, the substrate is prepared using paper process technology, and the resulting sheet may be further reduced into cut rag or strips for inserting into the substrate-containing segment of an aerosol delivery device. In one embodiment, cast sheet technology may be used to make a flat sheet. The cast sheet generally comprises a binder material, an inert filler, and one or more aerosol formers. The cast sheet can, in some embodiments, further comprise a component-containing, alginate-based substrate, as provided herein, such that such entrapped component(s) are included within the substrate. Optionally, wood derived fibers, a botanical, an active ingredient, and/or tobacco or a tobacco-derived material may be added during manufacture of the substrate as described below.

For example, in some embodiments the fibrous material, one or more aerosol forming materials, and a binder may be blended together to form a slurry, which may be cast onto a surface (such as, for example, a moving belt). Any one of these components (and/or additional components, as provided above) can, in some embodiments, be provided in the form of component-containing, alginate-based substrates). The cast slurry may then experience one or more drying and/or doctoring steps such that the result in a relatively consistent thickness cast sheet. Other examples of casting and paper-making techniques are set forth in U.S. Pat. No. 4,674,519 to Keritsis et al.; U.S. Pat. No. 4,941,484 to Clapp et al.; U.S. Pat. No. 4,987,906 to Young et al.; U.S. Pat. No. 4,972,854 to Kiernan et al.; U.S. Pat. No. 5,099,864 to Young et al.; U.S. Pat. No. 5,143,097 to Sohn et al.; U.S. Pat. No. 5,159,942 to Brinkley et al.; U.S. Pat. No. 5,322,076 to Brinkley et al.; U.S. Pat. No. 5,339,838 to Young et al.; U.S. Pat. No. 5,377,698 to Litzinger et al.; U.S. Pat. No. 5,501,237 to Young; and U.S. Pat. No. 6,216,706 to Kumar; the disclosures of which is incorporated herein by reference in their entireties. In some embodiments, the flat sheet may further be reduced into cut rag or strips for inserting into the substrate-containing segment of an aerosol delivery device. The cast sheet may also be gathered or rolled into a rod for insertion into the substrate-containing segment of an aerosol delivery device.

In another embodiment, the substrate may be prepared by granular extrusion followed by spheronization or marumerization to produce round or ovoid shaped beads, or hair-like rods. The granular extrusion formulation may be similar to that of the cast sheet formulation, except, for example, that an alternate or additional binder (e.g., a cellulose derivative) may be used therein. In yet another embodiment, the substrate may be prepared by extrusion followed by cutting or sizing to provide multiple size and/or shaped substrate pieces. The extrusion formulation may be similar to that of the granular extrusion formulation, except, for example, that yet another binder or binder combination (e.g., a combination of cellulose derivatives) may be used therein.

In any of the previous embodiments, the entire quantity of aerosol forming materials may be added prior to casting, extrusion, or the like, to form the aerosol generating component as disclosed herein. In certain embodiments, the entire quantity or a portion of aerosol forming materials may be provided by the component-containing, alginate-based substrates provided herein. Alternatively, or in addition, a portion or all of the aerosol forming materials may be impregnated into the substrate post-formation (e.g., one or more aerosol forming materials may be sprayed or otherwise disposed in or on the substrate material to form the aerosol generating component as disclosed herein.

In some embodiments, the substrate may comprise a plant-derived non-tobacco material, including, but not limited to, hemp, flax, sisal, rice straw, esparto, and/or a cellulose pulp material. In some instances, processed substrates can be employed as longitudinally extending strands. See, for example, the type of configuration set forth in U.S. Pat. No. 5,025,814 to Raker, which is incorporated herein by reference in its entirety. In addition, certain types of substrates can be formed, rolled, or gathered into a desired configuration. In still other implementations, the substrate material may comprise inorganic fibers of various types (e.g., fiber glass, metal wires/screens, etc.) and/or (organic) synthetic polymers. In various implementations, these “fibrous” materials could be unstructured (e.g., randomly distributed like the cellulose fibers in tobacco cast sheet) or structured (e.g., a wire mesh). In some embodiments, the substrate comprises, on a weight basis, from about 0 to about 5% of wood fibers or wood-derived fibers, for example, about 0%, about 1%, about 2%, about 3%, about 4%, or about 5% wood fibers or wood-derived fibers.

In some embodiments, the substrate may further comprise a burn retardant material, conductive fibers or particles for heat conduction/induction, or any combination thereof. One example of a burn retardant material is ammonium phosphate. In some embodiments, other flame/burn retardant materials and additives may be included within the substrate, and may include organo-phosphorus compounds, borax, hydrated alumina, graphite, potassium, silica, tripolyphosphate, dipentaerythritol, pentaerythritol, and polyols. Other burn retardant materials, such as nitrogenous phosphonic acid salts, mono-ammonium phosphate, ammonium polyphosphate, ammonium bromide, ammonium borate, ethanol-ammonium borate, ammonium sulphamate, halogenated organic compounds, thiourea, and antimony oxides may be incorporated into the substrates of the present disclosure. In each aspect of flame-retardant, burn-retardant, and/or scorch-retardant materials used in the substrate, the desirable properties are independent of and resistant to undesirable off-gassing or melting-type behavior. Various manners and methods for incorporating materials into smoking articles, and particularly smoking articles that are designed so as to not purposefully burn, are set forth in U.S. Pat. No. 4,947,874 to Brooks et al.; U.S. Pat. No. 7,647,932 to Cantrell et al.; U.S. Pat. No. 8,079,371 to Robinson et al.; U.S. Pat. No. 7,290,549 to Banerjee et al.; and U.S. Pat. App. Pub. No. 2007/0215167 to Crooks et al.; the disclosures of which are incorporated herein by reference in their entireties.

As noted, the substrate may also include conductive fibers or particles for heat conduction or heating by induction. In some embodiments, the conductive fibers or particles may be arranged in a substantially linear and parallel pattern. In some embodiments, the conductive fibers or particles may have a substantially random arrangement. In some embodiments, the conductive fibers or particles may be constructed of one or more of an aluminum material, a stainless steel material, a copper material, a carbon material, and a graphite material. In some embodiments, one or more conductive fibers or particles with different Curie temperatures may be included in the substrate material to facilitate heating by induction at varying temperatures.

In some embodiments, the substrate further comprises one or more additional components, which can vary in type and amounts thereof. For example, substrates can comprise, e.g., binders, fillers, tobacco materials, active ingredients, non-tobacco botanicals, flavorants, a nicotine component, or any combination thereof. Examples of suitable such components include, e.g., cellulose derivatives, starches, gums (e.g., xanthan gum, guar gum, gum Arabic, locust bean gum, and gum tragacanth), dextrans, carrageenan, calcium carbonate, etc. and further examples of suitable such components are described herein above with reference to the component-containing, alginate-based substrates. Further guidance regarding materials that may be provided within substrates of the type provided herein are described, for example, in U.S. Pat. No. 10,201,187, which is incorporated herein by reference in its entirety. It is noted that the component-containing, alginate-based substrates provided herein may, in some embodiments, fulfill one or more of the functions desired in a substrate when incorporated within the substrate. Thus, a substrate can comprise a component-containing, alginate-based substrate, wherein the “components” therein comprise any one or more of the components outlined herein as advantageously included within a substrate. In some embodiments, further components can be provided within the substrate independently (i.e., not within the component-containing, alginate-based substrate).

### Aerosol Delivery Devices

FIG. 5 illustrates a perspective schematic view of an aerosol generating component according to an example embodiment of the disclosure. In particular, FIG. 6 illustrates the aerosol generating component 104 having a substrate portion 110, and this aerosol generating component is an example of a consumable according to the disclosure. With reference to the description above, in the depicted embodiment, the substrate portion 110 can, in some embodiments, comprise one or more component-containing, alginate-based substrates in addition to and/or in replacement of the typical components of such a substrate, as provided herein above. In various embodiments, the term “overlapping layers” may also include bunched, crumpled, crimped, and/or otherwise gathered layers in which the individual layers may not be obvious.

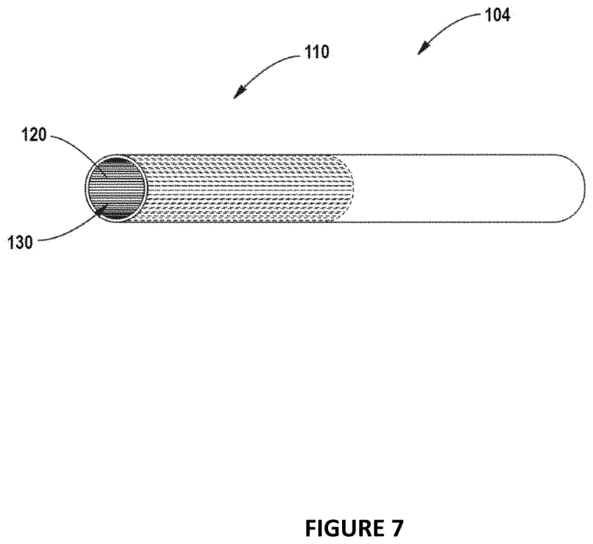

For example, FIG. 7 illustrates a schematic cross-section drawing of a substrate portion of an aerosol generating component according to an example embodiment of the present disclosure. In particular, FIG. 7 illustrates the substrate portion 110, which comprises a series of overlapping layers 130 of the substrate sheet 120. In the depicted embodiment, at least a portion of the overlapping layers 130 is substantially surrounded about its outer surface with a first cover layer 132. Although in various embodiments the composition of the first cover layer 132 may vary, in the depicted embodiment the first cover layer 132 comprises a combination of a fibrous material, the aerosol forming materials, and a binder material. Reference is made to the discussions herein relating possible aerosol forming materials and binder materials. In various embodiments, the first cover layer 132 may be constructed via a casting process, such as that described in U.S. Pat. No. 5,697,385 to Seymour et al., the disclosure of which is incorporated herein by reference in its entirety.

In the depicted embodiment, at least a portion of the overlapping layers 130 and the first cover layer 132 are substantially surrounded about an outer surface with a second cover layer 134. Although the composition of the second cover layer 134 may vary, in the depicted embodiment the second cover layer 134 comprises a metal foil material, such as an aluminum foil material. In other embodiments, the second cover layer may comprise other materials, including, but not limited to, a copper material, a tin material, a gold material, an alloy material, a ceramic material, or other thermally conductive amorphous carbon-based material, and/or any combinations thereof. The depicted embodiment further includes a third cover layer 136, which substantially surrounds the overlapping layers 130, first cover layer 132, and the second cover layer 134, about an outer surface thereof. In the depicted embodiment, the third cover layer 136 comprises a paper material, such as a conventional cigarette wrapping paper. In various embodiments, the paper material may comprise rag fibers, such as non-wood plant fibers, and may include flax, hemp, sisal, rice straw, and/or esparto fibers.

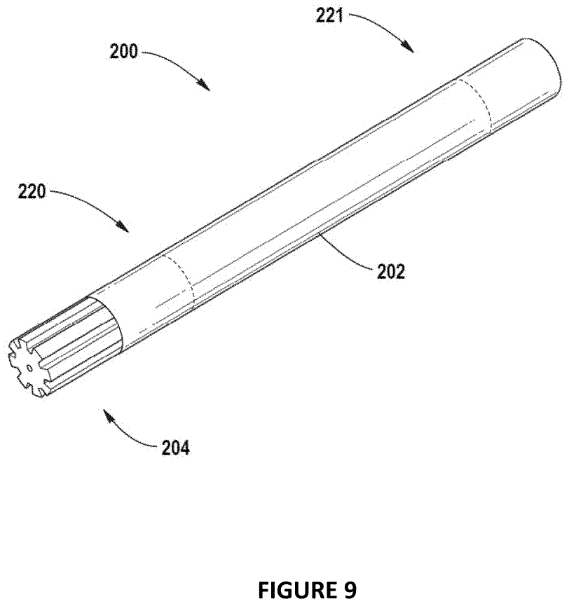

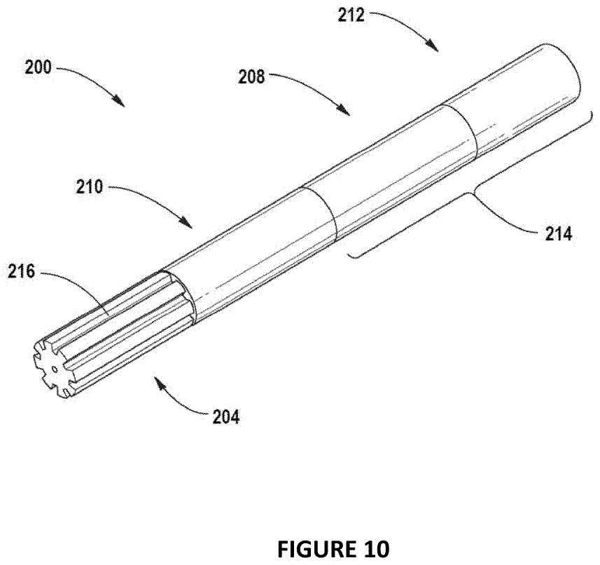

FIG. 9 illustrates a perspective view of an aerosol generating component, according to another example embodiment of the present disclosure, and FIG. 10 illustrates a perspective view of the aerosol generating component of FIG. 9 with an outer wrap removed. In particular, FIG. 9 illustrates an aerosol generating component 200 that includes an outer wrap 202, and FIG. 10 illustrates the aerosol generating component 200 wherein the outer wrap 202 is removed to reveal the other components of the aerosol generating component 200. In the depicted embodiment, the aerosol generating component 200 of the depicted embodiment includes a heat source 204, a substrate portion 210, an intermediate component 208, and a filter 212. In the depicted embodiment, the intermediate component 208 and the filter 212 together comprise a mouthpiece 214.

Although an aerosol delivery device and/or an aerosol generating component according to the present disclosure may take on a variety of embodiments, as discussed in detail below, the use of the aerosol delivery device and/or aerosol generating component by a consumer will be similar in scope. The foregoing description of use of the aerosol delivery device and/or aerosol generating component is applicable to the various embodiments described through minor modifications, which are apparent to the person of skill in the art in light of the further disclosure provided herein. The description of use, however, is not intended to limit the use of the articles of the present disclosure but is provided to comply with all necessary requirements of disclosure herein.

In various embodiments, the heat source 204 may be configured to generate heat upon ignition thereof. In the depicted embodiment, the heat source 204 comprises a combustible fuel element that has a generally cylindrical shape and that incorporates a combustible carbonaceous material. In other embodiments, the heat source 204 may have a different shape, for example, a prism shape having a triangular, cubic or hexagonal cross-section. Carbonaceous materials generally have a high carbon content. Preferred carbonaceous materials may be composed predominately of carbon, and/or typically may have carbon contents of greater than about 60 percent, generally greater than about 70 percent, often greater than about 80 percent, and frequently greater than about 90 percent, on a dry weight basis.

In some instances, the heat source 204 may incorporate elements other than combustible carbonaceous materials (e.g., tobacco components, such as powdered tobaccos or tobacco extracts; flavoring agents; salts, such as sodium chloride, potassium chloride and sodium carbonate; heat stable graphite fibers; iron oxide powder; glass filaments; powdered calcium carbonate; alumina granules; ammonia sources, such as ammonia salts; binding agents, such as guar gum, ammonium alginate and sodium alginate; and/or phase change materials for lowering the temperature of the heat source, described herein above). Although specific dimensions of an applicable heat source may vary, in some embodiments, the heat source 204 may have a length in an inclusive range of approximately 7 mm to approximately 20 mm, and in some embodiments may be approximately 17 mm, and an overall diameter in an inclusive range of approximately 3 mm to approximately 8 mm, and in some embodiments may be approximately 4.8 mm (and in some embodiments, approximately 7 mm).

Although in other embodiments, the heat source may be constructed in a variety of ways, in the depicted embodiment, the heat source 204 is extruded or compounded using a ground or powdered carbonaceous material, and has a density that is greater than about 0.5 g/cm3, often greater than about 0.7 g/cm3, and frequently greater than about 1 g/cm3, on a dry weight basis. See, for example, the types of fuel source components, formulations and designs set forth in U.S. Pat. No. 5,551,451 to Riggs et al. and U.S. Pat. No. 7,836,897 to Borschke et al., which are incorporated herein by reference in their entireties.

Although in various embodiments, the heat source may have a variety of forms, including, for example, a substantially solid cylindrical shape or a hollow cylindrical (e.g., tube) shape, the heat source 204 of the depicted embodiment comprises an extruded monolithic carbonaceous material that has a generally cylindrical shape but with a plurality of grooves 216 extending longitudinally from a first end of the extruded monolithic carbonaceous material to an opposing second end of the extruded monolithic carbonaceous material. In some embodiments, the aerosol delivery device, and in particular, the heat source, may include a heat transfer component. In various embodiments, a heat transfer component may be proximate the heat source, and, in some embodiments, a heat transfer component may be located in or within the heat source. Some examples of heat transfer components are described in in U.S. Pat. App. Pub. No. 2019-0281891 to Hejazi et al., which is incorporated herein by reference in its entirety.

Although in the depicted embodiment, the grooves 216 of the heat source 204 are substantially equal in width and depth and are substantially equally distributed about a circumference of the heat source 204, other embodiments may include as few as two grooves, and still other embodiments may include as few as a single groove. Still other embodiments may include no grooves at all. Additional embodiments may include multiple grooves that may be of unequal width and/or depth, and which may be unequally spaced around a circumference of the heat source. In still other embodiments, the heat source may include flutes and/or slits extending longitudinally from a first end of the extruded monolithic carbonaceous material to an opposing second end thereof. In some embodiments, the heat source may comprise a foamed carbon monolith formed in a foam process of the type disclosed in U.S. Pat. No. 7,615,184 to Lobovsky, which is incorporated herein by reference in its entirety. As such, some embodiments may provide advantages with regard to reduced time taken to ignite the heat source. In some other embodiments, the heat source may be co-extruded with a layer of insulation (not shown), thereby reducing manufacturing time and expense. Other embodiments of fuel elements include carbon fibers of the type described in U.S. Pat. No. 4,922,901 to Brooks et al. or other heat source embodiments such as is disclosed in U.S. Pat. App. Pub. No. 2009/0044818 to Takeuchi et al., each of which is incorporated herein by reference in its entirety.

Generally, the heat source is positioned sufficiently near an aerosol generating component (e.g., a substrate portion) having one or more aerosolizable components so that the aerosol formed/volatilized by the application of heat from the heat source to the aerosolizable components (as well as any flavorants, medicaments, and/or the like that are likewise provided for delivery to a user) is deliverable to the user by way of the mouthpiece. That is, when the heat source heats the substrate portion, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof. Additionally, the selection of various aerosol delivery device elements is appreciated upon consideration of commercially available electronic aerosol delivery devices, such as those representative products listed in the background art section of the present disclosure.

Referring back to FIGS. 9 and 10, the outer wrap 202 may be provided to engage or otherwise join together at least a portion of the heat source 204 with the substrate portion 210 and at least a portion of the mouthpiece 214. In various embodiments, the outer wrap 202 is configured to be retained in a wrapped position in any manner of ways including via an adhesive, or a fastener, and the like, to allow the outer wrap 202 to remain in the wrapped position. Otherwise, in some other aspects, the outer wrap 202 may be configured to be removable as desired. For example, upon retaining the outer wrap 202 in a wrapped position, the outer wrap 202 may be able to be removed from the heat source 204, the substrate portion 210, and/or the mouthpiece 214.

In some embodiments, in addition to the outer wrap 202, the aerosol delivery device may also include a liner that is configured to circumscribe the substrate portion 210 and at least a portion of the heat source 204. Although in other embodiments the liner may circumscribe only a portion of the length of the substrate portion 210, in some embodiments, the liner may circumscribe substantially the full length of the substrate portion 210. In some embodiments, the outer wrap material 202 may include the liner. As such, in some embodiments the outer wrap material 202 and the liner may be separate materials that are provided together (e.g., bonded, fused, or otherwise joined together as a laminate). In other embodiments, the outer wrap 202 and the liner may be the same material. In any event, the liner may be configured to thermally regulate conduction of the heat generated by the ignited heat source 204, radially outward of the liner. As such, in some embodiments, the liner may be constructed of a metal foil material, an alloy material, a ceramic material, or other thermally conductive amorphous carbon-based material, and/or an aluminum material, and in some embodiments may comprise a laminate. In some embodiments, depending on the material of the outer wrap 202 and/or the liner, a thin layer of insulation may be provided radially outward of the liner. Thus, the liner may advantageously provide, in some aspects, a manner of engaging two or more separate components of the aerosol generating component 200 (such as, for example, the heat source 204, the substrate portion 210, and/or a portion of the mouthpiece 214), while also providing a manner of facilitating heat transfer axially there along, but restricting radially outward heat conduction.

As shown in FIG. 9, the outer wrap 202 (and, as necessary, the liner, and the substrate portion 210) may also include one or more openings formed therethrough that allow the entry of air upon a draw on the mouthpiece 214. In various embodiments, the size and number of these openings may vary based on particular design requirements. In the depicted embodiment, a plurality of openings 220 are located proximate an end of the substrate portion 210 closest to the heat source 204, and a plurality of separate cooling openings 221 are formed in the outer wrap 202 (and, in some embodiments, the liner) in an area proximate the filter 212 of the mouthpiece 214. Although other embodiments may differ, in the depicted embodiment, the openings 220 comprise a plurality of openings substantially evenly spaced about the outer surface of the aerosol generating component 200, and the openings 221 also comprise a plurality of openings substantially evenly spaced around the outer surface of the aerosol generating component 200. Although in various embodiments the plurality of openings may be formed through the outer wrap 202 (and, in some embodiments, the liner) in a variety of ways, in the depicted embodiment, the plurality of openings 220 and the plurality of separate cooling openings 221 are formed via laser perforation.

Referring back to FIG. 10, the aerosol generating component 200 of the depicted implementation also includes an intermediate component 208 and at least one filter 212. It should be noted that in various implementations, the intermediate component 208 or the filter 212, individually or together, may be considered a mouthpiece 214 of the aerosol generating component 200. Although in various implementations, neither the intermediate component nor the filter need be included, in the depicted implementation the intermediate component 208 comprises a substantially rigid member that is substantially inflexible along its longitudinal axis. In the depicted implementation, the intermediate component 208 comprises a hollow tube structure, and is included to add structural integrity to the aerosol generating component 200 and provide for cooling the produced aerosol. In some implementations, the intermediate component 208 may be used as a container for collecting the aerosol. In various implementations, such a component may be constructed from any of a variety of materials and may include one or more adhesives. Example materials include, but are not limited to, paper, paper layers, paperboard, plastic, cardboard, and/or composite materials. In the depicted implementation, the intermediate component 208 comprises a hollow cylindrical element constructed of a paper or plastic material (such as, for example, ethyl vinyl acetate (EVA), or other polymeric materials such as poly ethylene, polyester, silicone, etc. or ceramics (e.g., silicon carbide, alumina, etc.), or other acetate fibers), and the filter comprises a packed rod or cylindrical disc constructed of a gas permeable material (such as, for example, cellulose acetate or fibers such as paper or rayon, or polyester fibers).

As noted, in some implementations the mouthpiece 214 may comprise a filter 212 configured to receive the aerosol therethrough in response to the draw applied to the mouthpiece 214. In various implementations, the filter 212 is provided, in some aspects, as a circular disc radially and/or longitudinally disposed proximate the second end of the intermediate component 208. In this manner, upon draw on the mouthpiece 214, the filter 212 receives the aerosol flowing through the intermediate component 208 of the aerosol generating component 200. In some implementations, the filter 212 may comprise discrete segments. For example, some implementations may include a segment providing filtering, a segment providing draw resistance, a hollow segment providing a space for the aerosol to cool, a segment providing increased structural integrity, other filter segments, and any one or any combination of the above. In some implementations, the filter 212 may additionally or alternatively contain strands of tobacco containing material, such as described in U.S. Pat. No. 5,025,814 to Raker et al., which is incorporated herein by reference in its entirety.

In various implementations the size and shape of the intermediate component 208 and/or the filter 212 may vary, for example the length of the intermediate component 208 may be in an inclusive range of approximately 10 mm to approximately 30 mm, the diameter of the intermediate component 208 may be in an inclusive range of approximately 3 mm to approximately 8 mm, the length of the filter 212 may be in an inclusive range of approximately 10 mm to approximately 20 mm, and the diameter of the filter 212 may be in an inclusive range of approximately 3 mm to approximately 8 mm. In the depicted implementation, the intermediate component 208 has a length of approximately 20 mm and a diameter of approximately 4.8 mm (and in some implementations, approximately 7 mm), and the filter 212 has a length of approximately 15 mm and a diameter of approximately 4.8 mm (or in some implementations, approximately 7 mm).

In various implementations, ignition of the heat source 204 results in aerosolization of the aerosol forming materials associated with the substrate portion 210. Preferably, the elements of the substrate portion 210 do not experience thermal decomposition (e.g., charring, scorching, or burning) to any significant degree, and the aerosolized components are entrained in the air that is drawn through the aerosol generating component 200, including the filter 212, and into the mouth of the user. In various implementations, the mouthpiece 214 (e.g., the intermediate component 208 and/or the filter 212) is configured to receive the generated aerosol therethrough in response to a draw applied to the mouthpiece 214 by a user. In some implementations, the mouthpiece 214 may be fixedly engaged to the substrate portion 210. For example, an adhesive, a bond, a weld, and the like may be suitable for fixedly engaging the mouthpiece 214 to the substrate portion 210. In one example, the mouthpiece 214 is ultrasonically welded and sealed to an end of the substrate portion 210. An example electrically-powered aerosol delivery device that can be used with substrates incorporating a component-containing, alginate-based substrate of the present disclosure is now described. In some embodiments, aerosol delivery devices may comprise some combination of a power source (e.g., an electrical power source), at least one control component (e.g., means for actuating, controlling, regulating and ceasing power for heat generation, such as by controlling electrical current flow from the power source to other components of the article, e.g., a microprocessor, individually or as part of a microcontroller), a heat source (e.g., an electrical resistance heating element or other component and/or an inductive coil or other associated components and/or one or more radiant heating elements), and an aerosol generating component that includes the disclosed substrates, which are capable of yielding an aerosol upon application of sufficient heat.

Note that it is possible to physically combine one or more of the above-noted components. For instance, in certain embodiments, a conductive heater trace can be printed on the surface of a substrate material as described herein (e.g., a nano-cellulose substrate film) using a conductive ink such that the heater trace can be powered by the power source and used as the resistance heating element. Example conductive inks include graphene inks and inks containing various metals, such as inks including silver, gold, palladium, platinum, and alloys or other combinations thereof (e.g., silver-palladium or silver-platinum inks), which can be printed on a surface using processes such as gravure printing, flexographic printing, off-set printing, screen printing, ink-jet printing, or other appropriate printing methods.

In various embodiments, a number of these components may be provided within an outer body or shell, which, in some embodiments, may be referred to as a housing. The overall design of the outer body or shell may vary, and the format or configuration of the outer body that may define the overall size and shape of the aerosol delivery device may vary. Although other configurations are possible, in some embodiments an elongated body resembling the shape of a cigarette or cigar may be a formed from a single, unitary housing or the elongated housing can be formed of two or more separable bodies. For example, an aerosol delivery device may comprise an elongated shell or body that may be substantially tubular in shape and, as such, resemble the shape of a conventional cigarette or cigar. In one example, all of the components of the aerosol delivery device are contained within one housing or body. In other embodiments, an aerosol delivery device may comprise two or more housings that are joined and are separable. For example, an aerosol delivery device may possess at one end a control body comprising a housing containing one or more reusable components (e.g., an accumulator such as a rechargeable battery and/or rechargeable super-capacitor, and various electronics for controlling the operation of that article), and at the other end and removably coupleable thereto, an outer body or shell containing a disposable portion (e.g., a disposable flavor-containing aerosol generating component).

In other embodiments, aerosol generating components of the present disclosure may generally include an ignitable heat source configured to heat the substrate material, as described above. The substrate material and/or at least a portion of the heat source may be covered in an outer wrap, or wrapping, a casing, a component, a module, a member, or the like. The overall design of the enclosure is variable, and the format or configuration of the enclosure that defines the overall size and shape of the aerosol generating component is also variable. Although other configurations are possible, it may be desirable, in some aspects, that the overall design, size, and/or shape of these embodiments resemble that of a conventional cigarette or cigar.

In this regard, FIG. 5 illustrates an aerosol delivery device 100 according to an example embodiment of the present disclosure. The aerosol delivery device 100 may include a control body 102 and an aerosol generating component 104. In various embodiments, the aerosol generating component 104 and the control body 102 may be permanently or detachably aligned in a functioning relationship. In this regard, FIG. 5 illustrates the aerosol delivery device 100 in a coupled configuration, whereas FIG. 6 illustrates the aerosol delivery device 100 in a decoupled configuration. Various mechanisms may connect the aerosol generating component 104 to the control body 102 to result in, for example, a threaded engagement, a press-fit engagement, an interference fit, a sliding fit, a magnetic engagement, or the like.

In various embodiments, the aerosol delivery device 100 according to than example embodiment of the present disclosure may have a variety of overall shapes, including, but not limited to an overall shape that may be defined as being substantially rod-like or substantially tubular shaped or substantially cylindrically shaped. In the embodiments of FIGS. 5 and 6, the device 100 has a substantially round cross-section; however, other cross-sectional shapes (e.g., oval, square, triangle, etc.) also are encompassed by the present disclosure. For example, in some embodiments one or both of the control body 102 or the aerosol generating component 104 (and/or any subcomponents) may have a substantially rectangular shape, such as a substantially rectangular cuboid shape (e.g., similar to a USB flash drive). In other embodiments, one or both of the control body 102 or the aerosol generating component 104 (and/or any subcomponents) may have other hand-held shapes. For example, in some embodiments the control body 102 may have a small box shape, various pod mod shapes, or a fob-shape. Thus, such language that is descriptive of the physical shape of the article may also be applied to the individual components thereof, including the control body 102 and the aerosol generating component 104.

Alignment of the components within the aerosol delivery device of the present disclosure may vary across embodiments. In some embodiments, the substrate portion may be positioned proximate a heat source so as to maximize aerosol delivery to the user. Other configurations, however, are not excluded. Generally, the heat source may be positioned sufficiently near the substrate portion so that heat from the heat source can volatilize the substrate portion (as well as, in some embodiments, one or more flavorants, medicaments, or the like that may likewise be provided for delivery to a user) and form an aerosol for delivery to the user. When the heat source heats the substrate portion, an aerosol is formed, released, or generated in a physical form suitable for inhalation by a consumer. It should be noted that the foregoing terms are meant to be interchangeable such that reference to release, releasing, releases, or released includes form or generate, forming or generating, forms or generates, and formed or generated. Specifically, an inhalable substance is released in the form of a vapor or aerosol or mixture thereof, wherein such terms are also interchangeably used herein except where otherwise specified.

As noted above, the aerosol delivery device 100 of various embodiments may incorporate a battery and/or other electrical power source to provide current flow sufficient to provide various functionalities to the aerosol delivery device, such as powering of the heat source, powering of control systems, powering of indicators, and the like. The power source may take on various configurations. Preferably, the power source may be able to deliver sufficient power to rapidly activate the heat source to provide for aerosol formation and power the aerosol delivery device through use for a desired duration of time. In some embodiments, the power source is sized to fit conveniently within the aerosol delivery device so that the aerosol delivery device can be easily handled. Examples of useful power sources include lithium-ion batteries that are preferably rechargeable (e.g., a rechargeable lithium-manganese dioxide battery). In particular, lithium polymer batteries can be used as such batteries can provide increased safety. Other types of batteries—e.g., N50-AAA CADNICA nickel-cadmium cells—may also be used. Additionally, a preferred power source is of a sufficiently light weight to not detract from a desirable smoking experience. Some examples of possible power sources are described in U.S. Pat. No. 9,484,155 to Peckerar et al., and U.S. Pat. App. Pub. No. 2017/0112191 to Sur et al., the disclosures of which are incorporated herein by reference in their respective entireties.

In specific embodiments, one or both of the control body 102 and the aerosol generating component 104 may be referred to as being disposable or as being reusable. For example, the control body 102 may have a replaceable battery or a rechargeable battery, solid-state battery, thin-film solid-state battery, rechargeable super-capacitor or the like, and thus may be combined with any type of recharging technology, including connection to a wall charger, connection to a car charger (i.e., cigarette lighter receptacle), and connection to a computer, such as through a universal serial bus (USB) cable or connector (e.g., USB 2.0, 3.0, 3.1, USB Type-C), connection to a photovoltaic cell (sometimes referred to as a solar cell) or solar panel of solar cells, a wireless charger, such as a charger that uses inductive wireless charging (including for example, wireless charging according to the Qi wireless charging standard from the Wireless Power Consortium (WPC)), or a wireless radio frequency (RF) based charger. An example of an inductive wireless charging system is described in U.S. Pat. App. Pub. No. 2017/0112196 to Sur et al., which is incorporated herein by reference in its entirety. Further, in some embodiments, the aerosol generating component 104 may comprise a single-use device. A single use component for use with a control body is disclosed in U.S. Pat. No. 8,910,639 to Chang et al., which is incorporated herein by reference in its entirety.

In further embodiments, the power source may also comprise a capacitor. Capacitors are capable of discharging more quickly than batteries and can be charged between puffs, allowing the battery to discharge into the capacitor at a lower rate than if it were used to power the heat source directly. For example, a super-capacitor—e.g., an electric double-layer capacitor (EDLC)—may be used separate from, or in combination with, a battery. When used alone, the super-capacitor may be recharged before each use of the article. Thus, the device may also include a charger component that can be attached to the smoking article between uses to replenish the super-capacitor.

Further components may be utilized in the aerosol delivery device of the present disclosure. For example, the aerosol delivery device may include a flow sensor that is sensitive either to pressure changes or air flow changes as the consumer draws on the article (e.g., a puff-actuated switch). Other possible current actuation/deactuation mechanisms may include a temperature actuated on/off switch or a lip pressure actuated switch. An example mechanism that can provide such puff-actuation capability includes a Model 163PC01D36 silicon sensor, manufactured by the MicroSwitch division of Honeywell, Inc., Freeport, Ill. Representative flow sensors, current regulating components, and other current controlling components including various microcontrollers, sensors, and switches for aerosol delivery devices are described in U.S. Pat. No. 4,735,217 to Gerth et al., U.S. Pat. Nos. 4,922,901, 4,947,874, and 4,947,875, all to Brooks et al., U.S. Pat. No. 5,372,148 to McCafferty et al., U.S. Pat. No. 6,040,560 to Fleischhauer et al., U.S. Pat. No. 7,040,314 to Nguyen et al., and U.S. Pat. No. 8,205,622 to Pan, all of which are incorporated herein by reference in their entireties. Reference is also made to the control schemes described in U.S. Pat. No. 9,423,152 to Ampolini et al., which is incorporated herein by reference in its entirety.

In another example, an aerosol delivery device may comprise a first conductive surface configured to contact a first body part of a user holding the device, and a second conductive surface, conductively isolated from the first conductive surface, configured to contact a second body part of the user. As such, when the aerosol delivery device detects a change in conductivity between the first conductive surface and the second conductive surface, a vaporizer is activated to vaporize a substance so that the vapors may be inhaled by the user holding unit. The first body part and the second body part may be a lip or parts of a hand(s). The two conductive surfaces may also be used to charge a battery contained in the personal vaporizer unit. The two conductive surfaces may also form, or be part of, a connector that may be used to output data stored in a memory. Reference is made to U.S. Pat. No. 9,861,773 to Terry et al., which is incorporated herein by reference in its entirety.

In addition, U.S. Pat. No. 5,154,192 to Sprinkel et al. discloses indicators for smoking articles; U.S. Pat. No. 5,261,424 to Sprinkel, Jr. discloses piezoelectric sensors that can be associated with the mouth-end of a device to detect user lip activity associated with taking a draw and then trigger heating of a heating device; U.S. Pat. No. 5,372,148 to McCafferty et al. discloses a puff sensor for controlling energy flow into a heating load array in response to pressure drop through a mouthpiece; U.S. Pat. No. 5,967,148 to Harris et al. discloses receptacles in a smoking device that include an identifier that detects a non-uniformity in infrared transmissivity of an inserted component and a controller that executes a detection routine as the component is inserted into the receptacle; U.S. Pat. No. 6,040,560 to Fleischhauer et al. describes a defined executable power cycle with multiple differential phases; U.S. Pat. No. 5,934,289 to Watkins et al. discloses photonic-optronic components; U.S. Pat. No. 5,954,979 to Counts et al. discloses means for altering draw resistance through a smoking device; U.S. Pat. No. 6,803,545 to Blake et al. discloses specific battery configurations for use in smoking devices; U.S. Pat. No. 7,293,565 to Griffen et al. discloses various charging systems for use with smoking devices; U.S. Pat. No. 8,402,976 to Fernando et al. discloses computer interfacing means for smoking devices to facilitate charging and allow computer control of the device; U.S. Pat. No. 8,689,804 to Fernando et al. discloses identification systems for smoking devices; and PCT Pat. App. Pub. No. WO 2010/003480 by Flick discloses a fluid flow sensing system indicative of a puff in an aerosol generating system; all of the foregoing disclosures being incorporated herein by reference in their entireties.

Further examples of components related to electronic aerosol delivery articles and disclosing materials or components that may be used in the present device include U.S. Pat. No. 4,735,217 to Gerth et al.; U.S. Pat. No. 5,249,586 to Morgan et al.; U.S. Pat. No. 5,666,977 to Higgins et al.; U.S. Pat. No. 6,053,176 to Adams et al.; U.S. Pat. No. 6,164,287 to White; U.S. Pat. No. 6,196,218 to Voges; U.S. Pat. No. 6,810,883 to Felter et al.; U.S. Pat. No. 6,854,461 to Nichols; U.S. Pat. No. 7,832,410 to Hon; U.S. Pat. No. 7,513,253 to Kobayashi; U.S. Pat. No. 7,896,006 to Hamano; U.S. Pat. No. 6,772,756 to Shayan; U.S. Pat. Nos. 8,156,944 and 8,375,957 to Hon; U.S. Pat. No. 8,794,231 to Thorens et al.; U.S. Pat. No. 8,851,083 to Oglesby et al.; U.S. Pat. Nos. 8,915,254 and 8,925,555 to Monsees et al.; U.S. Pat. No. 9,220,302 to DePiano et al.; U.S. Pat. App. Pub. Nos. 2006/0196518 and 2009/0188490 to Hon; U.S. Pat. App. Pub. No. 2010/0024834 to Oglesby et al.; U.S. Pat. App. Pub. No. 2010/0307518 to Wang; PCT Pat. App. Pub. No. WO 2010/091593 to Hon; and PCT Pat. App. Pub. No. WO 2013/089551 to Foo, each of which is incorporated herein by reference in its entirety.

Further, U.S. Pat. App. Pub. No. 2017/0099877 to Worm et al., filed Oct. 13, 2015, discloses capsules that may be included in aerosol delivery devices and fob-shape configurations for aerosol delivery devices, and is incorporated herein by reference in its entirety. A variety of the materials disclosed by the foregoing documents may be incorporated into the present devices in various embodiments, and all of the foregoing disclosures are incorporated herein by reference in their entireties.

Referring to FIG. 6, in the depicted embodiment, the aerosol generating component 104 comprises a heated end 106, which is configured to be inserted into the control body 102, and a mouth end 108, upon which a user draws to create the aerosol. At least a portion of the heated end 106 may include the previously described substrate portion 110. In various embodiments, the mouth end 108 of the aerosol generating component 104 may include a filter 114, which may, for example, be made of a cellulose acetate or polypropylene material. The filter 114 may additionally or alternatively contain strands of tobacco containing material, such as described in U.S. Pat. No. 5,025,814 to Raker et al., which is incorporated herein by reference in its entirety. In various embodiments, the filter 114 may increase the structural integrity of the mouth end of the aerosol source member, and/or provide filtering capacity, if desired, and/or provide resistance to draw. In some embodiments, the filter may comprise discrete segments. For example, some embodiments may include a segment providing filtering, a segment providing draw resistance, a hollow segment providing a space for the aerosol to cool, a segment providing increased structural integrity, other filter segments, and any one or any combination of the above.

In some embodiments, the material of the exterior overwrap 112 may comprise a material that resists transfer of heat, which may include a paper or other fibrous material, such as a cellulose material. The exterior overwrap material may also include at least one filler material imbedded or dispersed within the fibrous material. In various embodiments, the filler material may have the form of water insoluble particles. Additionally, the filler material may incorporate inorganic components. In various embodiments, the exterior overwrap may be formed of multiple layers, such as an underlying, bulk layer and an overlying layer, such as a typical wrapping paper in a cigarette. Such materials may include, for example, lightweight “rag fibers” such as flax, hemp, sisal, rice straw, and/or esparto. The exterior overwrap may also include a material typically used in a filter element of a conventional cigarette, such as cellulose acetate. Further, an excess length of the exterior overwrap at the mouth end 108 of the aerosol generating component may function to simply separate the substrate portion 110 from the mouth of a consumer or to provide space for positioning of a filter material, as described below, or to affect draw on the article or to affect flow characteristics of the vapor or aerosol leaving the device during draw. Further discussions relating to the configurations for exterior overwrap materials that may be used with the present disclosure may be found in U.S. Pat. No. 9,078,473 to Worm et al., which is incorporated herein by reference in its entirety.

In various embodiments, other components may exist between the substrate portion 110 and the mouth end 108 of the aerosol generating component 104. For example, in some embodiments one or any combination of the following may be positioned between the substrate portion 110 and the mouth end 108 of the aerosol generating component 104: an air gap; a hollow tube structure; phase change materials for cooling air; flavor releasing media; ion exchange fibers capable of selective chemical adsorption; aerogel particles as filter medium; and other suitable materials. Some examples of possible phase change materials include, but are not limited to, salts, such as AgNO3, AlCl3, TaCl3, InCl3, SnCl2, AlI3, and TiI4; metals and metal alloys such as selenium, tin, indium, tin-zinc, indium-zinc, or indium-bismuth; and organic compounds such as D-mannitol, succinic acid, p-nitrobenzoic acid, hydroquinone and adipic acid. Other examples are described in U.S. Pat. No. 8,430,106 to Potter et al., which is incorporated herein by reference in its entirety.

As will be discussed in more detail below, the presently disclosed aerosol generating component is configured for use with a conductive and/or inductive heat source to heat the substrate material to form an aerosol. In various embodiments, a conductive heat source may comprise a heating assembly that comprises a resistive heating member. Resistive heating members may be configured to produce heat when an electrical current is directed therethrough. Electrically conductive materials useful as resistive heating members may be those having low mass, low density, and moderate resistivity and that are thermally stable at the temperatures experienced during use. Useful heating members heat and cool rapidly, and thus provide for the efficient use of energy. Rapid heating of the member may be beneficial to provide almost immediate volatilization of an aerosol forming materials in proximity thereto. Rapid cooling prevents substantial volatilization (and hence waste) of the aerosol forming materials during periods when aerosol formation is not desired. Such heating members may also permit relatively precise control of the temperature range experienced by the aerosol forming materials, especially when time based current control is employed. Useful electrically conductive materials are preferably chemically non-reactive with the materials being heated (e.g., aerosol forming materials and other inhalable substance materials) so as not to adversely affect the flavor or content of the aerosol or vapor that is produced. Some example, non-limiting, materials that may be used as the electrically conductive material include carbon, graphite, carbon/graphite composites, metals, ceramics such as metallic and non-metallic carbides, nitrides, oxides, silicides, inter-metallic compounds, cermets, metal alloys, and metal foils. In particular, refractory materials may be useful. Various, different materials can be mixed to achieve the desired properties of resistivity, mass, and thermal conductivity. In specific embodiments, metals that can be utilized include, for example, nickel, chromium, alloys of nickel and chromium (e.g., nichrome), and steel. Materials that can be useful for providing resistive heating are described in U.S. Pat. No. 5,060,671 to Counts et al.; U.S. Pat. No. 5,093,894 to Deevi et al.; U.S. Pat. No. 5,224,498 to Deevi et al.; U.S. Pat. No. 5,228,460 to Sprinkel Jr., et al.; U.S. Pat. No. 5,322,075 to Deevi et al.; U.S. Pat. No. 5,353,813 to Deevi et al.; U.S. Pat. No. 5,468,936 to Deevi et al.; U.S. Pat. No. 5,498,850 to Das; U.S. Pat. No. 5,659,656 to Das; U.S. Pat. No. 5,498,855 to Deevi et al.; U.S. Pat. No. 5,530,225 to Hajaligol; U.S. Pat. No. 5,665,262 to Hajaligol; U.S. Pat. No. 5,573,692 to Das et al.; and U.S. Pat. No. 5,591,368 to Fleischhauer et al., the disclosures of which are incorporated herein by reference in their entireties.

In various embodiments, a heating member may be provided in a variety of forms, such as in the form of a foil, a foam, a mesh, a hollow ball, a half ball, discs, spirals, fibers, wires, films, yarns, strips, ribbons, or cylinders. Such heating members often comprise a metal material and are configured to produce heat as a result of the electrical resistance associated with passing an electrical current therethrough. Such resistive heating members may be positioned in proximity to, and/or in direct contact with, the substrate portion. For example, in one embodiment, a heating member may comprise a cylinder or other heating device located in the control body 102, wherein the cylinder is constructed of one or more conductive materials, including, but not limited to, copper, aluminum, platinum, gold, silver, iron, steel, brass, bronze, carbon (e.g., graphite), or any combination thereof. In various embodiments, the heating member may also be coated with any of these or other conductive materials. The heating member may be located proximate an engagement end of the control body 102, and may be configured to substantially surround a portion of the heated end 106 of the aerosol generating component 104 that includes the substrate portion 110. In such a manner, the heating member may be located proximate the substrate portion 110 of the aerosol generating component 104 when the aerosol source member is inserted into the control body 102. In other examples, at least a portion of a heating member may penetrate at least a portion of an aerosol generating component (such as, for example, one or more prongs and/or spikes that penetrate an aerosol generating component), when the aerosol generating component is inserted into the control body. Although in some embodiments the heating member may comprise a cylinder, it should be noted that in other embodiments, the heating member may take a variety of forms and, in some embodiments, may make direct contact with and/or penetrate the substrate portion.

As described above, in addition to being configured for use with a conductive heat source, the present disclosure may also be configured for use with an inductive heat source to heat the substrate portion to form an aerosol. In various embodiments, an inductive heat source may comprise a resonant transformer, which may comprise a resonant transmitter and a resonant receiver (e.g., a susceptor). In some embodiments, the resonant transmitter and the resonant receiver may be located in the control body 102. In other embodiments, the resonant receiver, or a portion thereof, may be located in the aerosol source member 104. For example, in some embodiments, the control body 102 may include a resonant transmitter, which, for example, may comprise a foil material, a coil, a cylinder, or other structure configured to generate an oscillating magnetic field, and a resonant receiver, which may comprise one or more prongs that extend into the substrate portion or are surrounded by the substrate portion. In some embodiments, the aerosol generating component is in intimate contact with the resonant receiver.

In other embodiments, a resonant transmitter may comprise a helical coil configured to circumscribe a cavity into which an aerosol generating component, and in particular, a substrate portion of an aerosol generating component, is received. In some embodiments, the helical coil may be located between an outer wall of the device and the receiving cavity. In one embodiment, the coil winds may have a circular cross section shape; however, in other embodiments, the coil winds may have a variety of other cross section shapes, including, but not limited to, oval shaped, rectangular shaped, L-shaped, T-shaped, triangular shaped, and combinations thereof. In another embodiment, a pin may extend into a portion of the receiving cavity, wherein the pin may comprise the resonant transmitter, such as by including a coil structure around or within the pin. In various embodiments, an aerosol source member may be received in the receiving cavity wherein one or more components of the aerosol source member may serve as the resonant receiver. In some embodiments, the aerosol generating component comprises the resonant receiver. Other possible resonant transformer components, including resonant transmitters and resonant receivers, are described in U.S. Pat. No. 10,517,332 to Sebastian et al., which is incorporated herein by reference in its entirety.

Although in some embodiments an aerosol generating component and a control body may be provided together as a complete smoking article or pharmaceutical delivery article generally, the components may be provided separately. For example, the present disclosure also encompasses a disposable unit for use with a reusable smoking article or a reusable pharmaceutical delivery article. In specific embodiments, such a disposable unit (which may be an aerosol generating component as illustrated in the appended figures) can comprise a substantially tubular shaped body having a heated end configured to engage the reusable smoking article or pharmaceutical delivery article, an opposing mouth end configured to allow passage of an inhalable substance to a consumer, and a wall with an outer surface and an inner surface that defines an interior space. Various embodiments of an aerosol generating component (or cartridge) are described in U.S. Pat. No. 9,078,473 to Worm et al., which is incorporated herein by reference in its entirety.

Although some figures described herein illustrate the control body and aerosol generating component in a working relationship, it is understood that the control body and the aerosol generating component may exist as individual devices. Accordingly, any discussion otherwise provided herein in relation to the components in combination also should be understood as applying to the control body and the aerosol generating component as individual and separate components.

Although the component-containing, alginate-based substrates provided herein may, in some embodiments, be advantageously incorporated within the types of devices outlined above, it is noted that their use is not limited thereto.

### Oral Products

In some embodiments, component-containing, alginate-based substrates are incorporated within products configured for oral use. The term “configured for oral use” as used herein means that the product is provided in a form such that during use, saliva in the mouth of the user causes one or more of the components of the composition (e.g., flavoring agents and/or active ingredients) to pass into the mouth of the user. In certain embodiments, the product is adapted to deliver components to a user through mucous membranes in the user's mouth, the user's digestive system, or both, and, in some instances, said component is an active ingredient (including, but not limited to, for example, a stimulant, vitamin, taste modifier, or combination thereof) that can be absorbed through the mucous membranes in the mouth or absorbed through the digestive tract when the product is used. In some embodiments, products configured for oral use comprise a nicotine component. Any of the components of an oral product (including, e.g., flavorants, active ingredients, nicotine component, sweeteners, etc.) can optionally be provided in the form of a component-containing, alginate-based substrate.

Products configured for oral use as described herein may take various forms, including gels, gummies, pastilles, gums, lozenges, powders, beverages, beads, meltable products, and pouches. Gels can be soft or hard. Certain products configured for oral use are in the form of pastilles. As used herein, the term “pastille” refers to a dissolvable oral product made by solidifying a liquid or gel composition so that the final product is a somewhat hardened solid gel. The rigidity of the gel is highly variable. Certain products of the disclosure are in the form of solids. Certain products can exhibit, for example, one or more of the following characteristics: crispy, granular, chewy, syrupy, pasty, fluffy, smooth, and/or creamy. In certain embodiments, the desired textural property can be selected from the group consisting of adhesiveness, cohesiveness, density, dryness, fracturability, graininess, gumminess, hardness, heaviness, moisture absorption, moisture release, mouthcoating, roughness, slipperiness, smoothness, viscosity, wetness, and combinations thereof.

In other embodiments, products configured for oral use are in the form of a composition disposed within a moisture-permeable container (e.g., a water-permeable pouch). Such compositions in the water-permeable pouch format are typically used by placing one pouch containing the composition in the mouth of a human subject/user. Generally, the pouch is placed somewhere in the oral cavity of the user, for example under the lips, in the same way as moist snuff products are generally used. The pouch preferably is not chewed or swallowed. Exposure to saliva then causes some of the components of the composition therein (e.g., flavoring agents and/or active ingredients) to pass through e.g., the water-permeable pouch and provide the user with flavor and satisfaction, and the user is not required to spit out any portion of the composition. After about 10 minutes to about 60 minutes, typically about 15 minutes to about 45 minutes, of use/enjoyment, substantial amounts of the composition have been absorbed through oral mucosa of the human subject, and the pouch may be removed from the mouth of the human subject for disposal.

Various types of products configured for oral use (into which the disclosed component-containing, alginate-based substrates can be incorporated) are described, e.g., in U.S. Pat. No. 5,167,244 to Kjerstad and U.S. Pat. No. 8,931,493 to Sebastian et al.; as well as US Patent App. Pub. No. 2008/0196730 to Engstrom et al.; 2008/0305216 to Crawford et al.; 2009/0293889 to Kumar et al.; 2010/0291245 to Gao et al; 2011/0139164 to Mua et al.; 2012/0037175 to Cantrell et al., 2012/0055494 to Hunt et al.; 2012/0138073 to Cantrell et al.; 2012/0138074 to Cantrell et al.; 2013/0074855 to Holton, Jr.; 2013/0074856 to Holton, Jr.; 2013/0152953 to Mua et al.; 2013/0274296 to Jackson et al.; 2015/0068545 to Moldoveanu et al.; 2015/0101627 to Marshall et al.; 2015/0230515 to Lampe et al.; 2016/0000140 to Sebastian et al.; 2016/0073689 to Sebastian et al.; 2016/0157515 to Chapman et al.; and 2016/0192703 to Sebastian et al., which are all incorporated herein by reference in their entireties.

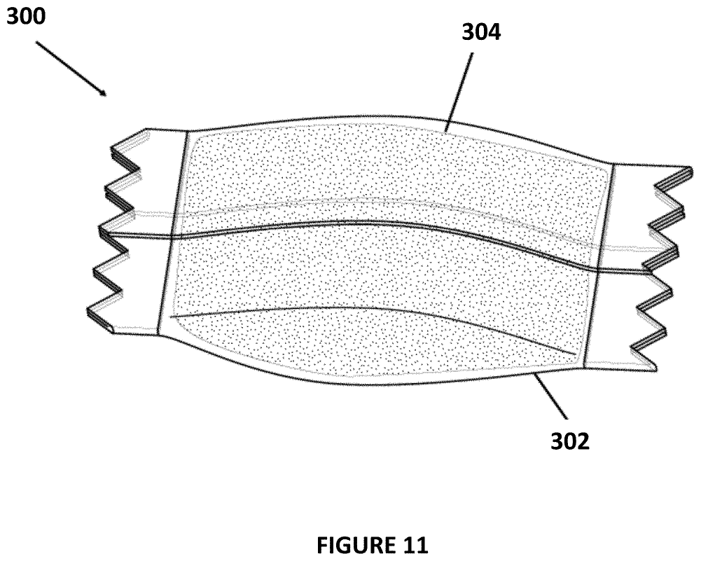

In some embodiments, a pouched product is provided, which generally comprises a pouch at least partially filled with a composition configured for oral use. The pouch can, in some embodiments, be constructed of a component-containing, cross-linked alginate-based substrate as provided herein. Referring to FIG. 11, there is shown a first embodiment of a pouched product 300. The pouched product 300 includes a moisture-permeable container in the form of a pouch 302, which can be formed of a component-containing alginate-based substrate as provided herein, and which contains a material 304 comprising a composition for oral use. In some embodiments, a smokeless product is provided wherein the container is formed of an alginate-based substrate as provided herein. In such embodiments, once the user has enjoyed the oral composition or other smokeless tobacco composition provided therein, the user can chew and ingest the pouch/container, instead of spitting out and/or discarding the emptied remains.

In various embodiments, inclusion of a component-containing alginate-based substrate as provided herein within an oral product can provide for delayed and/or extended release of the component(s) entrapped therein. For example, in specific embodiments, incorporating an active agent within a component-containing alginate-based substrate and including that component-containing alginate-based substrate within an oral product can provide for extended release of the active agent within the oral cavity (e.g., about 5 to about 30 minutes, such as about 5 minutes or greater, about 10 minutes or greater, about 15 minutes or greater, about 20 minutes or greater, or even longer, such as about 30 minutes to about 6 hours (e.g., about 30 minutes or greater, about 45 minutes or greater, about 1 hour or greater, about 2 hours or greater, about 3 hours or greater, or about 4 hours or greater). The extended release can have various benefits in addition to simply extending the release of the active agent within the oral cavity; for example, where the active agent is associated with an unfavorable taste (e.g., including, but not limited to, bitterness) and/or an unfavorable sensation within the oral cavity (e.g., including, but not limited to, a burning feeling), extending the release of the active agent over a period of time can help to decrease such unfavorable characteristics. Similarly, in some embodiments, it may be advantageous to include a flavorant within a component-containing alginate-based substrate (alone or in combination with an active agent). In such embodiments, extended release of a flavorant may enhance the sensation within the oral cavity, more effectively masking negative flavors or sensations (e.g., including, but not limited to, bitterness and/or burning feelings associated with release of an active agent into the oral cavity). In some embodiments, delaying release of components via incorporation within a component-containing alginate-based substrate as provided herein can lead to release of at least a portion of such components (e.g., including all such components) at a later point in the digestion process, such as within the stomach or small intestine.

### Smoking Articles

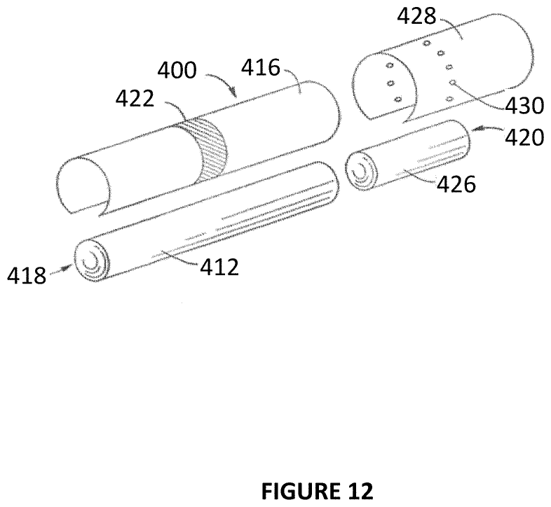

Furthermore, in some embodiments, the disclosed component-containing, alginate-based substrates can be incorporated within conventional smoking articles. In some such embodiments, the component-containing, alginate-based substrate is incorporated within the tobacco rod or filter element of a smoking article. The exact configuration and components of a smoking article can vary. Referring to FIG. 12, there is shown a smoking article 400 in the form of a cigarette and possessing certain representative components of a smoking article that can contain the formulation of the present invention. The cigarette 400 includes a generally cylindrical rod 412 of a charge or roll of smokable filler material (e.g., about 0.3 g to about 1.0 g of smokable filler material such as tobacco material) contained in a circumscribing wrapping material 416. The rod 412 is conventionally referred to as a “tobacco rod.” The ends of the tobacco rod 412 are open to expose the smokable filler material. The cigarette 410 is shown as having one optional band 422 (e.g., a printed coating including a film-forming agent, such as starch, ethylcellulose, or sodium alginate) applied to the wrapping material 416, and that band circumscribes the cigarette rod in a direction transverse to the longitudinal axis of the cigarette. The band 422 can be printed on the inner surface of the wrapping material (i.e., facing the smokable filler material), or less preferably, on the outer surface of the wrapping material.

At one end of the tobacco rod 412 is the lighting end 418, and at the mouth end 420 is positioned a filter element 426. The filter element 426 positioned adjacent one end of the tobacco rod 412 such that the filter element and tobacco rod are axially aligned in an end-to-end relationship, preferably abutting one another. Filter element 426 may have a generally cylindrical shape, and the diameter thereof may be essentially equal to the diameter of the tobacco rod. The ends of the filter element 426 permit the passage of air and smoke therethrough. A ventilated or air diluted smoking article can be provided with an optional air dilution means, such as a series of perforations 430, each of which extend through the tipping material and plug wrap. The optional perforations 430 can be made by various techniques known to those of ordinary skill in the art, such as laser perforation techniques. Alternatively, so-called off-line air dilution techniques can be used (e.g., through the use of porous paper plug wrap and pre-perforated tipping paper). The component-containing, alginate-based substrates provided herein can be incorporated within any of the components of a smoking article, including but not limited to, as a component of the tobacco charge, as a component of the wrapping paper (e.g., as the paper or coated on the interior or exterior of the paper), as an adhesive, as a filter element component, and/or within a capsule located in any region of the smoking article (e.g., a crushable capsule in the filter of a tobacco rod).

Having now described some illustrative embodiments of the invention, it should be apparent to those skilled in the art that the foregoing is merely illustrative and not limiting, having been presented by way of example only. Numerous modifications and other embodiments are within the scope of one of ordinary skill in the art and are contemplated as falling within the scope of the invention. In particular, although many of the examples presented herein involve specific combinations of method steps or system elements, it should be understood that those steps and those elements may be combined in other ways to accomplish the same objectives.

Furthermore, those skilled in the art should appreciate that the parameters and configurations described herein are examples only and that actual parameters and/or configurations will depend on the specific application in which the systems and techniques of the invention are used. Those skilled in the art should also recognize or be able to ascertain, using no more than routine experimentation, equivalents to the specific embodiments of the invention. It is, therefore, to be understood that the embodiments described herein are presented by way of example only and that, within the scope of any appended claims and equivalents thereto; the invention may be practiced other than as specifically described.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

What is claimed is:

1. A method for providing a composition with a releasable entrapment of one or more components in a component-containing, cross-linked alginate structure, comprising:
mixing the one or more components and alginate in water to give a mixture;
contacting the mixture with a divalent or trivalent cation to crosslink the alginate, thereby trapping the one or more components within a cross-linked matrix; and
removing at least a portion of the water from the cross-linked matrix to give a component-containing alginate structure.

2. The method of claim 1, wherein the one or more components are selected from the group consisting of flavorants, sweeteners, aerosol-forming agents, humectants, fillers, preservatives, tobacco materials, and combinations thereof.

3. The method of claim 2, wherein the one or more components comprise a flavorant selected from the group consisting of alcohols, aldehydes, aromatic hydrocarbons, ketones, esters, terpenes, terpenoids, trigeminal sensates, and combinations thereof.

4. The method of claim 3, wherein the flavorant is selected from the group consisting of vanillin, ethyl vanillin, p-anisaldehyde, hexanal, furfural, isovaleraldehyde, cuminaldehyde, benzaldehyde, citronellal, 1-hydroxy-2-propanone, 2-hydroxy-3-methyl-2-cyclopentenone-1-one, allyl hexanoate, ethyl heptanoate, ethyl hexanoate, isoamyl acetate, 3-methylbutyl acetate, sabinene, limonene, gamma-terpinene, beta-farnesene, nerolidol, thujone, myrcene, geraniol, nerol, citronellol, linalool, eucalyptol, and combinations thereof.

5. The method of claim 2, wherein the one or more components comprise a flavorant selected from cream, tea, coffee, fruit, maple, menthol, mint, peppermint, spearmint, wintergreen, nutmeg, clove, lavender, cardamom, ginger, honey, anise, sage, rosemary, hibiscus, rose hip, Yerba mate, guayusa, honeybush, rooibos, Yerba santa, Bacopa monniera, Gingko biloba, Withania somnifera, cinnamon, sandalwood, jasmine, cascarilla, cocoa, licorice, and combinations thereof.

6. The method of claim 1, wherein the one or more components comprise a plant extract.

7. The method of claim 6, wherein the plant extract is a tobacco extract.

8. The method of claim 1, wherein the one or more components comprise an active ingredient, selected from the group consisting of a nicotine component, a botanical/herbal ingredient, a stimulant, an amino acid, a vitamin, an antioxidant, a cannabinoid, a cannabimimetic, a terpene, a pharmaceutical ingredient, and any combination thereof.

9. The method of claim 8, wherein the active ingredient is selected from the group consisting of hemp, guarana, eucalyptus, rooibos, fennel, citrus, cloves, lavender, peppermint, chamomile, basil, rosemary, ginger, turmeric, green tea, white mulberry, cannabis, cocoa, ashwagandha, baobab, chlorophyll, cordyceps, damiana, ginseng, guarana, maca, tisanes, lemon balm, ginseng, star anise, caffeine, theacrine, theobromine, theophylline, GABA, theanine, taurine, Vitamin B6, Vitamin B12, Vitamin E, Vitamin C, cannabidiol (CBD), tetrahydrocannabinol (THC), and any combination thereof.

10. The method of claim 1, wherein the one or more components comprise an aerosol forming agent, selected from the group consisting of a polyhydric alcohol, a sorbitan ester, a fatty acid, a wax, a terpene, and any combination thereof.

11. The method of claim 10, wherein the aerosol forming agent is selected from the group consisting of glycerol, propylene glycol, 1,3-propanediol, diethylene glycol, triethylene glycol, sorbitan monolaurate, sorbitan monostearate (Span 60), sorbitan monooleate (Span 20), sorbitan tristearate (Span 65), butyric acid, propionic acid, valeric acid, oleic acid, linoleic acid, stearic acid, myristic acid, palmitic acid, monolaurin, glycerol monostearate, triolein, tripalmitin, tristearate, glycerol tributyrate, glycerol trihexanoate, carnauba wax, beeswax, candellila, limonene, pinene, farnesene, myrcene, geraniol, fennel, cembrene, and any combination thereof.

12. The method of claim 1, wherein the one or more components comprise a flavorant, a filler, an aerosol-forming agent, or any combination thereof, and wherein the incorporating comprises incorporating the component-containing, cross-linked alginate structure as a substrate within a consumable portion of a non-combustible aerosol delivery device.

13. The method of claim 1, wherein the divalent or trivalent cations are selected from the group consisting of calcium (Ca²⁺), barium (Ba²⁺), magnesium (Mg²⁺), strontium (Sr²⁺) iron (Fe²⁺), aluminum (Al³⁺), and combinations thereof.

14. The method of claim 1, wherein the contacting step comprises depositing the mixture into a solution comprising the divalent or trivalent cation.

15. The method of claim 14, wherein a speed of depositing the mixture into the solution is controlled to form discrete shapes.

16. The method of claim 1, further comprising casting a sheet of the mixture, and wherein the contacting step comprises contacting the sheet with a solution comprising the divalent or trivalent cations.

17. The method of claim 16, further comprising cutting or shredding the component-containing, cross-linked alginate structure to provide strips.

18. The method of claim 1, wherein the contacting step comprises bringing the mixture into contact with an outer surface of a pre-formed structure, wherein the pre-formed structure comprises the divalent or trivalent cation.

19. The method of claim 18, wherein the pre-formed structure is a bead and the mixture is in the form of a coating on the outer surface of the bead.

20. The method of claim 1, wherein the component-containing, cross-linked alginate structure is in the form of a sheet, a strip, a bead, or a corkscrew-shaped noodle.

21. The method of claim 1, further comprising incorporating the component-containing, cross-linked alginate structure within a consumable product.

22. The method of claim 21, wherein the consumable product is selected from the group consisting of a product configured for combustible aerosol delivery, a product configured for non-combustible aerosol delivery, or a product configured for aerosol-free delivery.

23. A component-containing cross-linked alginate structure, comprising one or more components entrapped within a cross-linked alginate matrix.

24. The component-containing cross-linked alginate structure of claim 23, wherein the one or more components are selected from the group consisting of flavorants, sweeteners, aerosol-forming agents, humectants, fillers, preservatives, tobacco materials, and combinations thereof.

25. A consumable product selected from the group consisting of an aerosol delivery product, an oral product, and a conventional smoking article, comprising the component-containing cross-linked alginate structure of claim 23.

26. An aerosol generating component comprising a substrate carrying at least one aerosol forming material, the substrate comprising the component-containing cross-linked alginate structure of claim 24.

27. An aerosol delivery device, comprising:
the aerosol generating component of claim 26;
a heat source configured to heat the substrate carrying the one or more aerosol forming materials to form an aerosol; and
an aerosol pathway extending from the aerosol generating component to a mouth-end of the aerosol delivery device.
